(19) United States
(12) Patent Application Publication
(10) Pub. No.: US 2022/0017618 A1
(43) Pub. Date: Jan. 20, 2022

(54) USE OF TIM-3 ANTIBODY IN PREPARATION OF MEDICINES FOR TREATING TUMORS

(71) Applicants: Jiangsu Hengrui Medicine Co., Ltd., Lianyungang, Jiangsu (CN); Shanghai Hengrui Pharmaceutical Co., Ltd., Shanghai (CN)

(72) Inventors: Xing Sun, Lianyungang, Jiangsu (CN); Zhuoxiao Cao, Lianyungang, Jiangsu (CN); Zupeng Xu, Lianyungang, Jiangsu (CN); Cheng Liao, Lianyungang, Jiangsu (CN); Changyong Yang, Lianyungang, Jiangsu (CN); Lianshan Zhang, Lianyungang, Jiangsu (CN)

(21) Appl. No.: 17/266,450

(22) PCT Filed: Aug. 20, 2019
(86) PCT No.: PCT/CN2019/101552

§ 371 Date: Feb. 5, 2021

(30) Foreign Application Priority Data
Aug. 20, 2018 (CN) 201810946361.3

(51) Int. Cl. C07K 16/28; C07K 16/30; A61P 35/00

(52) U.S. Cl. CPC C07K 16/2818; C07K 16/3046; A61P 35/00; A61K 2039/505; C07K 2317/40; C07K 2317/52; C07K 2317/24

(57) ABSTRACT

Disclosed is use of a TIM-3 antibody in preparation of medicines for treating tumors. Specifically, provided is use of the TIM-3 antibody or an antigen-binding fragment thereof in preparation of medicines for treating non-small cell lung cancer, the TIM-3 antibody containing a heavy chain variable region shown in SEQ ID NO: 33 and a light chain variable region shown in SEQ ID NO: 36. Further, also provided is use of the TIM-3 antibody or the antigen-binding fragment thereof and a PD-1 antibody or an antigen-binding fragment thereof in joint preparation of medicines for treating tumors.

### CROSS-REFERENCE TO RELATED APPLICATIONS

This application is a U.S. National Phase of International PCT Application No. PCT/CN2019/101552 filed Aug. 20, 2019, which claims priority to Chinese Patent Application Serial No. 201810946361.3 filed Aug. 20, 2018, the contents of each application are incorporated herein by reference in their entirety.

### SEQUENCE LISTING

This application incorporates by reference the material in the ASCII text file titled English_Translation_of_Sequence_Listing.txt, which was created on Jan. 15, 2021 and is 76.4 KB.

### FIELD OF THE INVENTION

The present disclosure relates to the use of a TIM-3 antibody in the preparation of medicament for treating tumor.

### BACKGROUND OF THE INVENTION

T cell immunoglobulin mucin-domain-containing molecule 3 (TIM-3), also referred to as hepatitis A virus cellular receptor 2 (HAVCR-2), is Type I membrane surface protein, a member of TIM family. Human TIM-3 molecule is composed of 301 amino acids, comprising signal peptide, Ig variable region (IgV region), Ser/Thr-rich mucin region, trans-membrane region and cytoplasmic region; human TIM-3 shares 63% homology with murine TIM-3.

TIM-3 can regulate the function of the immune system in many ways. It can bind to ligand Gal-9 on the surface of Th1 cells to down-regulate Th1 cell response and induce Th1 cell apoptosis. It plays an important role in auto- and allogeneic immune diseases (such as systemic erythema lupus, asthma) and immune tolerance.

In addition, TIM-3 is not only expressed in immune cells, but also over-expressed in tumor cells such as ovarian cancer, meningioma, and melanoma, and directly promotes tumor growth. Down-regulating the expression of TIM-3 can significantly inhibit the invasion and metastasis of HeLa cells. The overexpression of TIM-3 is closely related to the poor prognosis of lung cancer, gastric cancer, prostate cancer and cervical cancer. In hematological tumors, TIM-3 is overexpressed on leukemia stem cells of acute myeloid leukemia and hematopoietic stem cells of MDS patients, and TIM-3+ hematopoietic stem cells have malignant biological characteristics such as low differentiation, low apoptosis and high proliferation. Therefore, inhibiting the activity of TIM-3 (such as TIM-3 antibody) to improve the function of the innate immune system is expected to become a new method for the treatment of tumors (see, for example, Ngiow et al., Cancer Res., 71(21): 1-5 (2011); Guo et al., Journal of Translational Medicine, 11: 215 (2013); and Ngiow et al., Cancer Res., 71(21): 6567-6571 (2011)).

At present, TIM-3 antibodies have been reported in several patent applications, such as WO2011159877, WO2013006490, WO2015117002, WO2016144803, WO2016161270, US20150218274.

WO2018153366 (application date February 26, 2018) describes a new TIM-3 antibody with high activity, excellent affinity and stability.

As a representative of tumor immunotherapy, the effect of PD-1 antibody is obvious. Clinical data has proved that PD-1 antibody can increase the 5-year survival rate from 17% to 34% for patients with malignant tumors, and from 4% to 16% for patients with non-small cell lung cancer. However, not all patients can benefit from PD-1 antibody, or PD-1 antibody does not work at all or only maintains a short-term effect.

It has been reported in Nature Communication (February 2016) that one of the reasons for the resistance to PD-1 antibodies is that tumors have developed a new immune escape pathway, TIM-3. In the study, EGFR (T790M/L858) and KRAS (G12D) mutant lung cancer mouse models were used to construct anti-PD-1 resistant mouse models respectively. The researchers firstly analyzed the change of number of T cells in mouse tumors before treatment and after resistance to anti-PD-1 treatment; and then specifically analyzed the relationship between TIM-3 positive expression and the resistance to anti-PD-1 treatment; and found that the TIM-3 positive expression is significantly time-dependent on the duration of anti-PD-1 treatment, the positive expression of TIM-3 is low before treatment and during the treatment-sensitive period, while the positive expression of TIM-3 increases significantly after the development of drug-resistance. TIM-3 positive expression is also significantly related to the binding degree of PD-1 antibody in T cells. The higher the degree of T cell binding to PD-1 antibody, the stronger the positive expression of TIM-3 is. In conclusion, the failure of anti-PD-1 therapy is related to the up-regulation of TIM-3 expression. This molecule promotes immune escape in a way similar to PD-1/L1 by inhibiting T cell function and promoting T cell failure (Nature volume 545, pages 60-65). In addition, two TIM-3 antibodies combined with PD-1 for the treatment of malignant tumors or advanced solid tumors are in the clinical research stage (NCT02608268 and NCT02817637). For this reason, the development of new TIM-3 antibody administered alone or in combination with PD-1 for the treatment of tumors has attracted sufficient interest from pharmaceutical researchers.

### SUMMARY OF THE INVENTION

The present disclosure provides use of a TIM-3 antibody in the preparation of a medicament for the treatment of tumors.

In some embodiments, the TIM-3 antibody or antigen-binding fragment thereof comprises one or more CDR region sequence(s) selected from the group consisting of: sequences of antibody heavy chain variable region HCDR: as shown in amino acid sequence SEQ ID NOs: 14, 15 and 16, or amino acid sequences having at least 95% sequence identity thereto; and sequences of antibody light chain variable region LCDR: as shown in amino acid sequence SEQ ID NOs: 17, 18 and 19, or amino acid sequences having at least 95% sequence identity thereto.

In some embodiments, the CDR sequences in the light and heavy chain of the TIM-3 antibody are shown in the following table:

| | | | |
|---|---|---|---|
| HCDR1 | DYYMA | LCDR1 | RASDNIYSYLA |
| | SEQ ID NO: 14 | | SEQ ID NO: 17 |
| HCDR2 | NINYDGSSTYYLDSLKS | LCDR2 | NAKTLAE |
| | SEQ ID NO: 15 | | SEQ ID NO: 18 |
| HCDR3 | DVGYYGGNYGFAY | LCDR3 | QQHYGSPLT |
| | SEQ ID NO: 16 | | SEQ ID NO: 19 |

In some embodiments, the TIM-3 antibody or antigen-binding fragment thereof comprises one or more CDR region sequence(s) selected from the group consisting of: sequences of antibody heavy chain variable region HCDR: as shown in amino acid sequence SEQ ID NOs: 8, 43 and 10, or amino acid sequences having at least 95% sequence identity thereto; and sequences of antibody light chain variable region LCDR: as shown in amino acid sequence SEQ ID NOs: 11, 12 and 13, or amino acid sequences having at least 95% sequence identity thereto, wherein the SEQ ID NO: 43 is shown in the sequence of DIIPX₁X₂X₃GSKYNQKFKD: wherein, X₁ is selected from N, L, V, M or E, X2 is selected from N, E, M, H, K, L, A or V, and X₃ is selected from G or A.

In other embodiments, the CDR sequences in the light and heavy chain of the TIM-3 antibody are shown in the following table:

| Heavy chain | | Light chain | |
|---|---|---|---|
| HCDR1 | DYYMN | LCDR1 | LASQPIGIWLA |
| | SEQ ID | | SEQ ID |
| | NO: 8 | | NO: 11 |
| HCDR2 | DIIPNNGGS | LCDR2 | AATSLAD |
| | KYNQKFKD | | SEQ ID |
| | SEQ ID | | NO: 12 |
| | NO: 9 | | |
| HCDR3 | WGYGSSYRWFDY | LCDR3 | QQLYSSPWT |
| | SEQ ID | | SEQ ID |
| | NO: 10 | | NO: 13 |

Preferably, in some embodiments, the TIM-3 antibody or antigen-binding fragment thereof is selected from the group consisting of murine antibody, chimeric antibody, humanized antibody or antigen-binding fragment thereof.

In some embodiments, the light chain and heavy chain FR region sequence(s) of the humanized antibody light chain and heavy chain variable region(s) is/are respectively derived from human germline light chain and heavy chain or the mutant sequence(s) thereof.

Further, in some embodiments, the humanized antibody comprises heavy chain variable region as shown in SEQ ID NO: 31 or variant thereof, and preferably the variant comprises 1 to 10 amino acid alternation(s) when compared with heavy chain variable region as shown in SEQ ID NO: 31, more preferably the amino acid alternations are amino acid back-mutations Q3K and R87K; and the humanized antibody comprises light chain variable region as shown in SEQ ID NO: 32 or variant thereof, and preferably the variant comprises 1 to 10 amino acid alternation(s) when compared with light chain variable region as shown in SEQ ID NO: 32, more preferably the amino acid alternation is selected from the group consisting of amino acid back-mutations Q3K and I48V, K45Q, A43S and T85S.

The sequences of the humanized antibody heavy and light chain variable region described above are as follows:

Sequence of heavy chain variable region,
SEQ ID NO: 31
EVQLVESGGGLVQPGGSLRLSCAASGFTFSDYYMAWVRQA
PGKGLEWVANINYDGSSTYYLDSLKSRFTISRDNAKNSLY
LQMNSLRAEDTAVYYCARDVGYYGGNYGFAYWGQGTLVTV
SS;
Sequence of light chain variable region,
SEQ ID NO: 32
DIQMTQSPSSLSASVGDRVTITCRASDNIYSYLAWYQQKP
GKAPKLLIYNAKTLAEGVPSRFSGSGSGTDFTLTISSLQP
EDFATYYCQQHYGSPLTFGQGTKLEIK;

Note: The arrangement is FR1-CDR1-FR2-CDR2-FR3-CDR3-FR4, the italics in the sequence represent the FR sequences, and the underline represents the CDR sequences.

In some embodiments, the sequences of the humanized TIM-3 antibody heavy and light chain variable regions are as follows:

heavy chain variable region sequence
SEQ ID NO: 33:
EVQLVESGGGLVQPGGSLRLSCAASGFTFSDYYMAWVRQAPGKGLEW
VANINYDGSSTYYLDSLKSRFTISRDNAKNSLYLQMNSLRAEDTAVY
YCARDVGYYGGNYGFAYWGQGTLVTVSS;
light chain variable region sequence
SEQ ID NO: 36:
DIQMTQSPSSLSASVGDRVTITCRASDNIYSYLAWYQQKPGKAPKLL
IYNAKTLAEGVPSRFSGSGSGTDFTLTISSLQPEDFATYYCQQHYGS
PLTFGQGTKLEIK.

In some embodiments, the humanized antibody comprises heavy chain variable region as shown in SEQ ID NO: 20 or variant thereof, and preferably the variant comprises 1 to 10 amino acid alternation(s) when compared with heavy chain variable region as shown in SEQ ID NO: 20, more preferably the amino acid alternations are amino acid back-mutations D89E, R98T, G49A, M48I, M70L, R38K and V68A; and the humanized antibody comprises light chain variable region as shown in SEQ ID NO: 21 or variant thereof, and preferably the variant comprises 1 to 10 amino acid alternation(s) when compared with light chain variable region as shown in SEQ ID NO: 21, more preferably the amino acid alternation is amino acid back-mutation A43S.

The sequences of the humanized antibody heavy and light chain variable regions described above are as follows:

heavy chain variable region sequence
SEQ ID NO: 20
QVQLVQSGAEVKKPGASVKVSCKASGYTFTDYYMNWVRQAPGQGLEWM
GDIIPNNGGSKYNQKFKDRVTMTTDTSTSTAYMELRSLRSDDTAVYYC
ARWGYGSSYRWFDYWGQGTLVTVSS;
light chain variable region sequence
SEQ ID NO: 21
DIQMTQSPSSLSASVGDRVTITCLASQPIGIWLAWYQQKPGKAPKLLI
YAATSLADGVPSRFSGSGSGTDFTFTISSLQPEDIATYYCQQLYSSPW
TFGGGTKVEIK;

Note: The arrangement is FR1-CDR1-FR2-CDR2-FR3-CDR3-FR4, the italics in the sequence represent the FR sequences, and the underline represents the CDR sequences.

In some embodiments, the sequences of the humanized TIM-3 antibody heavy and light chain variable regions are as follows:

heavy chain variable region sequence
SEQ ID NO: 51
QVQLVQSGAEVKKPGASVKVSCKASGYTFTDYYMNWVRQAPGQGLEWM
GDIIPNLGGSKYNQKFKDRVTMTTDTSTSTAYMELRSLRSDDTAVYYC
ATWGYGSSYRWFDYWGQGTLVTVSS;
light chain variable region sequence
SEQ ID NO: 29
DIQMTQSPSSLSASVGDRVTITCLASQPIGIWLAWYQQKPGKAPKLLI
YAATSLADGVPSRFSGSGSGTDFTFTISSLQPEDIATYYCQQLYSSPW
TFGGGTKVEIK.

Preferably, the TIM-3 antibody is a full-length antibody, further comprising human antibody constant region(s), preferably comprising human heavy chain constant region sequence as shown in SEQ ID NO: 41 and preferably human light chain constant region as shown in SEQ ID NO: 42.

The heavy chain constant region sequence is as
shown in SEQ ID NO: 41:
ASTKGPSVFPLAPCSRSTSESTAALGCLVKDYFPEPVTVSWNSGALTS
GVHTFPAVLQSSGLYSLSSVVTVPSSSLGTKTYTCNVDHKPSNTKVDK
RVESKYGPPCPPCPAPEFLGGPSVFLFPPKPKDTLMISRTPEVTCVVV
DVSQEDPEVQFNWYVDGVEVHNAKTKPREEQFNSTYRVVSVLTVLHQD
WLNGKEYKCKVSNKGLPSSIEKTISKAKGQPREPQVYTLPPSQEEMTK
NQVSLTCLVKGFYPSDIAVEWESNGQPENNYKTTPPVLDSDGSFFLYS
RLTVDKSRWQEGNVFSCSVMHEALHNHYTQKSLSLSLGK;
The light chain constant region sequence is as
shown in SEQ ID NO: 42:
RTVAAPSVFIFPPSDEQLKSGTASVVCLLNNFYPREAKVQWKVDNALQ
SGNSQESVTEQDSKDSTYSLSSTLTLSKADYEKHKVYACEVTHQGLSS
PVTKSFNRGEC.

In some embodiments, the antigen-binding fragment of the TIM-3 antibody is selected from the group consisting of Fab, Fab′, F(ab′)2, single-chain antibody (scFv), dimerized V region (diabody), disulfide bond stabilized V region (dsFv), and antigen-binding fragment of peptide containing CDRs.

In another aspect, the TIM-3 antibody or antigen-binding fragment thereof described in the use according to present disclosure is administered in combination with an anti-PD-1 antibody or antigen-binding fragment thereof.

Anti-PD-1 antibody is known and can be selected from but not limited to: AMP-224, GLS-010, IBI-308, REGN-2810, PDR-001, BGB-A317, Pidilizumab, PF-06801591, Genolimzumab, CA-170, MEDI-0680, JS-001, TSR-042, Camrelizumab, Pembrolizumab, LZM-009, AK-103 and Nivolumab.

Preferably, the light chain variable region of the PD-1 antibody comprises LCDR1, LCDR2 and LCDR3 as shown in SEQ ID NO: 76, SEQ ID NO: 77 and SEQ ID NO: 78, respectively; the heavy chain variable region of the anti-PD-1 antibody comprises HCDR1, HCDR2 and HCDR3 as shown in SEQ ID NO: 73, SEQ ID NO: 74 and SEQ ID NO: 75, respectively.

In other embodiments, each CDR sequence of the anti-PD-1 antibody is shown in the following table:

| Name | Sequence | SEQ ID NO |
|---|---|---|
| HCDR1 | SYMMS | SEQ ID NO: 73 |
| HCDR2 | TISGGGANTYYPDSVKG | SEQ ID NO: 74 |
| HCDR3 | QLYYFDY | SEQ ID NO: 75 |
| LCDR1 | LASQTIGTWLT | SEQ ID NO: 76 |
| LCDR2 | TATSLAD | SEQ ID NO: 77 |
| LCDR3 | QQVYSIPWT | SEQ ID NO: 78 |

Preferably, the anti-PD-1 antibody is a humanized antibody or fragment thereof.

In an alternative embodiment, the antigen-binding fragment of the anti-PD-1 antibody in the present disclosure is antibody fragment selected from the group consisting of Fab, Fab′-SH, Fv, scFv, and (Fab′) 2 fragment.

The immunoglobulin can be derived from any commonly known isotype, including but not limited to IgA, secreted IgA, IgG, and IgM. The subclasses of IgG are also well known to those skilled in the art, including but not limited to IgG1, IgG2, IgG3, and IgG4. “Isotype” refers to Ab class or subclass (for example, IgM or IgG1) encoded by the heavy chain constant region gene. In some alternative embodiments, the anti-PD-1 antibody or antigen-binding fragment thereof in the present disclosure comprises heavy chain constant region(s) of human IgG1, IgG2, IgG3, or IgG4 isotype, and preferably comprises heavy chain constant region(s) of IgG1 or IgG4 isotype.

In other alternative embodiments, the anti-PD-1 antibody or antigen-binding fragment thereof comprises light chain constant region(s) of kappa or lambda.

Further, preferably the sequence of the humanized antibody light chain variable region is the sequence as shown in SEQ ID NO: 82 or variant thereof, and preferably the variant has 0-10 amino acid alternation(s) in the light chain variable region, more preferably the amino acid alternation is A43 S; and the sequence of the humanized antibody heavy chain variable region is as shown in SEQ ID NO: 81 or variant thereof, and preferably the variant has 0-10 amino acid alternation(s) in the heavy chain variable region, more preferably the amino acid alternation is G44R.

In some embodiments, the sequences of the humanized anti-PD-1 antibody heavy and light chain variable regions are as follows:

Heavy chain variable region
SEQ ID NO: 81
EVQLVESGGGLVQPGGSLRLSCAASGFTFSSYMMSWVRQAPGKGLEWV
ATISGGGANTYYPDSVKGRFTISRDNAKNSLYLQMNSLRAEDTAVYYC
ARQLYYFDYWGQGTTVTVSS;
Light chain variable region
SEQ ID NO: 82
DIQMTQSPSSLSASVGDRVTITCLASQTIGTWLTWYQQKPGKAPKLLI
YTATSLADGVPSRFSGSGSGTDFTLTISSLQPEDFATYYCQQVYSIPW
TFGGGTKVEIK.

Preferably, the humanized anti-PD-1 antibody light chain sequence is the sequence as shown in SEQ ID NO: 80 or variant thereof; preferably the variant has 0-10 amino acid alternation(s) in the light chain variable region, more preferably the amino acid alternation is A43 S; the humanized antibody heavy chain sequence is the sequence as shown in SEQ ID NO: 79 or variant thereof, preferably the variant has 0-10 amino acid alternation(s) in the heavy chain variable region, more preferably the amino acid alternation is G44R.

In another embodiment, the light chain sequence of the humanized anti-PD-1 antibody is the sequence as shown in SEQ ID NO: 80, and the heavy chain sequence is the sequence as shown in SEQ ID NO: 79:

Heavy chain
SEQ ID NO: 79
EVQLVESGGGLVQPGGSLRLSCAASGFTFSSYMMSWVRQAPGKGLEWV
ATISGGGANTYYPDSVKGRFTISRDNAKNSLYLQMNSLRAEDTAVYYC
ARQLYYFDYWGQGTTVTVSSASTKGPSVFPLAPCSRSTSESTAALGCL
VKDYFPEPVTVSWNSGALTSGVHTFPAVLQSSGLYSLSSVVTVPSSSL
GTKTYTCNVDHKPSNTKVDKRVESKYGPPCPPCPAPEFLGGPSVFLFP
PKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAK
GQPREPQVYTLPPSQEEMTKNQVSLTCLVKGFYPSDIAVEWESNGQPE
NNYKTTPPVLDSDGSFFLYSRLTVDKSRWQEGNVFSCSVMHEALHNHY
TQKSLSLSLGK;
Light chain
SEQ ID NO: 80
DIQMTQSPSSLSASVGDRVTITCLASQTIGTWLTWYQQKPGKAPKLLI
YTATSLADGVPSRFSGSGSGTDFTLTISSLQPEDFATYYCQQVYSIPW
TFGGGTKVEIKRTVAAPSVFIFPPSDEQLKSGTASVVCLLNNFYPREA
KVQWKVDNALQSGNSQESVTEQDSKDSTYSLSSTLTLSKADYEKHKVY
ACEVTHQGLSSPVTKSFNRGEC.

The anti-PD-1 antibody combined with the TIM-3 antibody described in the present disclosure exhibits pharmaceutical synergistic effect in the preparation of medicament for the treatment of tumors.

Depending on the type and severity of the disease, the administration dosage in human subjects of the TIM-3 antibody or antigen-binding fragment thereof described herein (administered according to the weight of the patient) is 0.1 to 10.0 mg/kg, which can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.2 mg/kg, 1.4 mg/kg, 1.6 mg/kg, 1.8 mg/kg, 2.0 mg/kg, 2.2 mg/kg, 2.4 mg/kg, 2.6 mg/kg, 2.8 mg/kg, 3.0 mg/kg, 3.2 mg/kg, 3.4 mg/kg, 3.6 mg/kg, 3.8 mg/kg, 4.0 mg/kg, 4.2 mg/kg, 4.4 mg/kg, 4.6 mg/kg, 4.8 mg/kg, 5.0 mg/kg, 5.2 mg/kg, 5.4 mg/kg, 5.6 mg/kg, 5.8 mg/kg, 6.0 mg/kg, 6.2 mg/kg, 6.4 mg/kg, 6.6 mg/kg, 6.8 mg/kg, 7.0 mg/kg, 7.2 mg/kg, 7.4 mg/kg, 7.6 mg/kg, 7.8 mg/kg, 8.0 mg/kg, 8.2 mg/kg, 8.4 mg/kg, 8.6 mg/kg, 8.8 mg/kg, 9.0 mg/kg, 9.2 mg/kg, 9.4 mg/kg, 9.6 mg/kg, 9.8 mg/kg, 10.0 mg/kg.

In an alternative embodiment, the administration dosage in human subjects of the TIM-3 antibody or antigen-binding fragment thereof (administered according to the weight of the patient) is 1 mg to 1000 mg, which can be 1.0 mg, 1.2 mg, 1.4 mg, 1.6 mg, 1.8 mg, 2.0 mg, 2.2 mg, 2.4 mg, 2.6 mg, 2.8 mg, 3.0 mg, 3.2 mg, 3.4 mg, 3.6 mg, 3.8 mg, 4.0 mg, 4.2 mg, 4.4 mg, 4.6 mg, 4.8 mg, 5.0 mg, 5.2 mg, 5.4 mg, 5.6 mg, 5.8 mg, 6.0 mg, 6.2 mg, 6.4 mg, 6.6 mg, 6.8 mg, 7.0 mg, 7.2 mg, 7.4 mg, 7.6 mg, 7.8 mg, 8.0 mg, 8.2 mg, 8.4 mg, 8.6 mg, 8.8 mg, 9.0 mg, 9.2 mg, 9.4 mg, 9.6 mg, 9.8 mg, 10.0 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, preferably 50 to 600 mg, most preferably 200 mg.

The administration frequency will vary with the type and severity of the disease. In some embodiments, the administration frequency of the TIM-3 antibody or antigen-binding fragment thereof described in the present disclosure is once a week, once every two weeks, once every three weeks, once every four weeks, once every six weeks, or once every eight weeks.

In an alternative embodiment, the TIM-3 antibody or antigen-binding fragment thereof described in the present disclosure is administered in a human subject at a dosage of 50 to 600 mg/once every 2-3 weeks. However, other dosage may be useful, preferably 200 mg/once every 2-3 weeks.

The administration dosage in a human subject of the anti-PD-1 antibody or antigen-binding fragment thereof described herein is 0.1 to 10.0 mg/kg, which can be 0.1 mg/kg, 0.2 mg/kg, 0.3 mg/kg , 0.4 mg/kg, 0.5 mg/kg, 0.6 mg/kg, 0.7 mg/kg, 0.8 mg/kg, 0.9 mg/kg, 1.0 mg/kg, 1.2 mg/kg, 1.4 mg/kg, 1.6 mg/kg , 1.8 mg/kg, 2.0 mg/kg, 2.2 mg/kg, 2.4 mg/kg, 2.6 mg/kg, 2.8 mg/kg, 3.0 mg/kg, 3.2 mg/kg, 3.4 mg/kg, 3.6 mg/kg, 3.8 mg/kg, 4.0 mg/kg, 4.2 mg/kg, 4.4 mg/kg, 4.6 mg/kg, 4.8 mg/kg, 5.0 mg/kg, 5.2 mg/kg, 5.4 mg/kg, 5.6 mg/kg, 5.8 mg/kg, 6.0 mg/kg, 6.2 mg/kg, 6.4 mg/kg, 6.6 mg/kg, 6.8 mg/kg, 7.0 mg/kg, 7.2 mg/kg, 7.4 mg/kg, 7.6 mg/kg, 7.8 mg/kg, 8.0 mg/kg, 8.2 mg/kg, 8.4 mg/kg, 8.6 mg/kg, 8.8 mg/kg, 9.0 mg/kg, 9.2 mg/kg, 9.4 mg/kg, 9.6 mg/kg, 9.8 mg/kg, 10.0 mg/kg.

In an alternative embodiment, the administration dosage in a human subject of the anti-PD-1 antibody or antigen-binding fragment thereof is 1 mg to 1000 mg, which can be 1.0 mg, 1.2 mg, 1.4 mg, 1.6 mg, 1.8 mg, 2.0 mg, 2.2 mg, 2.4 mg, 2.6 mg, 2.8 mg, 3.0 mg, 3.2 mg, 3.4 mg, 3.6 mg, 3.8 mg, 4.0 mg, 4.2 mg, 4.4 mg, 4.6 mg, 4.8 mg, 5.0 mg, 5.2 mg, 5.4 mg, 5.6 mg, 5.8 mg, 6.0 mg, 6.2 mg, 6.4 mg, 6.6 mg, 6.8 mg, 7.0 mg, 7.2 mg, 7.4 mg, 7.6 mg, 7.8 mg, 8.0 mg, 8.2 mg, 8.4 mg, 8.6 mg, 8.8 mg, 9.0 mg, 9.2 mg, 9.4 mg, 9.6 mg, 9.8 mg, 10.0 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, 800 mg, 805 mg, 810 mg, 815 mg, 820 mg, 825 mg, 830 mg, 835 mg, 840 mg, 845 mg, 850 mg, 855 mg, 860 mg, 865 mg, 870 mg, 875 mg, 880 mg, 885 mg, 890 mg, 895 mg, 900 mg, 905 mg, 910 mg, 915 mg, 920 mg, 925 mg, 930 mg, 935 mg, 940 mg, 945 mg, 950 mg, 955 mg, 960 mg, 965 mg, 970 mg, 975 mg, 980 mg, 985 mg, 990 mg, 995 mg, 1000 mg, preferably 50 to 600 mg, most preferably 200 mg.

The administration frequency will vary with the type and severity of the disease. In some embodiments, the administration frequency of the anti-PD-1 antibody or antigen-binding fragment thereof described in the present disclosure is once a week, once every two weeks, once every three weeks, once every four weeks, once every six weeks, or once every eight weeks.

In an alternative embodiment, the anti-PD-1 antibody or antigen-binding fragment thereof described in the present disclosure is administered at a dosage of 50 to 600 mg/once every 2-3 weeks. However, other dosage may be useful, preferably 200 mg/once every 2-3 weeks.

In some embodiments, the administration dosage in a human subject of the TIM-3 antibody or antigen-binding fragment thereof (administered according to the weight of the patient) is 0.1 to 10.0 mg/kg, and administration dosage of the anti-PD-1 antibody or antigen-binding fragment thereof is 0.1 to 10.0 mg/kg.

In some embodiments, the administration dosage in a human subject of the TIM-3 antibody or antigen-binding fragment thereof is 1 to 1000 mg, and the administration dosage of the anti-PD-1 antibody or antigen-binding fragment thereof is 1 to 1000 mg, once every three weeks.

In some embodiments, the administration dosage in a human subject of the TIM-3 antibody or antigen-binding fragment thereof (administered according to the weight of the patient) is 1 to 1000 mg, and the administration dosage of the anti-PD-1 antibody or antigen-binding fragment thereof is 50 to 600 mg, once every three weeks.

In some embodiments, the administration dosage in a human subject of the TIM-3 antibody or antigen-binding fragment thereof (administered according to the weight of the patient) is 1 to 1000 mg, once every three weeks; and the administration dosage in a human subject of the anti-PD-1 antibody or antigen-binding fragment thereof (administered according to the weight of the patient) is 1 to 1000 mg.

The administration route in the present disclosure may be oral administration, parenteral administration, transdermal administration; the parenteral administration comprises but not limited to intravenous injection, subcutaneous injection, or intramuscular injection.

In a preferred embodiment of the present disclosure, the PD-1 antibody is administered by injection, such as subcutaneous or intravenous injection, and the PD-1 antibody must be formulated into an injectable form before injection. In particular, preferably the injectable form of the PD-1 antibody is injection solution or lyophilized powder, which comprises PD-1 antibody, buffer, stabilizer, and optionally surfactant. The buffer can be one or more selected from the group consisting of acetate, citrate, succinate and phosphate. The stabilizer may be selected from saccharides or amino acids, preferably disaccharides, such as sucrose, lactose, trehalose, and maltose. The surfactant is selected from the group consisting of polyoxyethylene hydrogenated castor oil, fatty acid glycerides, polyoxyethylene sorbitan fatty acid esters, preferably the polyoxyethylene sorbitan fatty acid ester is polysorbate 20, 40, 60 or 80, most preferably polysorbate 20. The most preferably injectable form of PD-1 antibody comprises PD-1 antibody, acetate buffer, trehalose and polysorbate 20.

The present disclosure also provides a pharmaceutical kit, or a pharmaceutical composition, which comprises the anti-TIM-3 antibody or antigen-binding fragment thereof and the anti-PD-1 antibody or antigen-binding fragment thereof.

The present disclosure also provides a method for treating tumors, comprising administering a therapeutically effective amount of the TIM-3 antibody or antigen-binding fragment thereof or/and the anti-PD-1 antibody or antigen-binding fragment thereof to a patient with tumor.

Examples of tumors described in the use of the present disclosure are selected from the group consisting of, but not limited to: breast cancer (such as triple negative breast cancer), lung cancer, gastric cancer, colorectal cancer (such as rectal cancer, colorectal cancer), kidney cancer (such as renal cell carcinoma), liver cancer (such as hepatocellular carcinoma), melanoma (such as metastatic melanoma), non-small cell lung cancer, lymphoblastic T-cell leukemia, chronic myeloid leukemia, chronic lymphocytic leukemia, hairy cell leukemia, acute lymphoblastic leukemia, acute myeloid leukemia (AML), chronic neutrophil leukemia, acute lymphoblastic T-cell leukemia, immunoblastic mast cell leukemia, mantle cell leukemia, multiple myeloma megakaryoblastic leukemia, acute megakaryocytic leukemia, promyelocytic leukemia, erythroleukemia, malignant lymphoma, multiple myeloma, plasmacytoma, Hodgkin's lymphoma, non-Hodgkin's lymphoma, lymphoblastic T-cell lymphoma, burkitt's lymphoma, follicular lymphoma and myelodysplastic syndrome (MDS).

In an alternative embodiment, the tumor in the use of the present disclosure is non-small cell lung cancer, breast cancer (such as triple negative breast cancer), melanoma (such as metastatic melanoma), kidney cancer, colorectal cancer or liver cancer, preferably colorectal cancer or non-small cell lung cancer.

Otherwise indicated specifically, the terms in the present disclosure have the following definition:

In the present disclosure, the so-called “in combination with” is a way of administration, which means that at least one dosage of the TIM-3 antibody or antigen-binding fragment thereof and at least one dosage of the anti-PD-1 antibody or antigen-binding fragment thereof are provided within given time period, in which both medicaments show pharmacological effect to produce pharmacological efficacy. This time period can be one dosing cycle. The two medicaments can be administered simultaneously or sequentially.

The “humanized antibody” used in the present disclosure, also known as CDR-grafted antibody, refers to an antibody generated by grafting mouse CDR sequences onto the human antibody variable region frameworks (i.e. antibodies produced within different types of human germline antibody framework sequences). Humanized antibodies overcome the strong antibody response induced by the chimeric antibody which carries a large amount of mouse protein components. Such framework sequences can be obtained from public DNA databases or published references that include germline antibody gene sequences. For example, the germline DNA sequences of human heavy chain and light chain variable region genes can be found in the “VBase” human germline sequence database (available on the Internet www.mrccpe.com.ac.uk/vbase), as well as in Kabat, E A, etc., 1991 Sequences of Proteins of Immunological Interest, 5th edition. In a preferred embodiment of the present disclosure, the CDR sequence of the PD-1 humanized antibody is selected from the group consisting of SEQ ID NO: 73, 74, 75, 76, 77 and 78.

The “murine antibody” used in the present disclosure is a monoclonal antibody against human TIM-3, which is prepared according to the knowledge and skills in the art. During the preparation, a test subject is injected with TIM-3 antigen, and then hybridoma expressing antibody which possesses desired sequences or functional characteristics is separated. In some preferred embodiments of the present invention, the murine TIM-3 antibody or antigen-binding fragment thereof further comprises light chain constant region(s) of murine κ, 80 , chain or variants thereof, or further comprises heavy chain constant region(s) of murine IgG1, IgG2, IgG3, or variants thereof.

The “chimeric antibody” used in the present disclosure is an antibody which is formed by fusing the variable region of a murine antibody with the constant region of a human antibody, the chimeric antibody can alleviate the murine antibody-induced immune response. To establish a chimeric antibody, hybridoma secreting specific murine monoclonal antibody is firstly established, a variable region gene is cloned from mouse hybridoma cells, then a constant region gene of a human antibody is cloned as desired, the mouse variable region gene is ligated to the human constant region gene to form a chimeric gene which can be then inserted into an expression vector, and finally the chimeric antibody molecule is expressed in an eukaryotic or prokaryotic system. In a preferred embodiment of the present invention, the antibody light chain of the TIM-3 chimeric antibody further comprises light chain constant region(s) of human κ, λ, chain or variant thereof. The antibody heavy chain of the TIM-3 chimeric antibody further comprises heavy chain constant region(s) of human IgG1, IgG2, IgG3, IgG4 or variant thereof, preferably comprises the human IgG1, IgG2 or IgG4 heavy chain constant region(s), or comprises IgG1, IgG2 or IgG4 variants comprising amino acid mutation(s) (such as YTE mutation or back-mutation).

The “antigen-binding fragment” of the anti-PD-1 antibody used in the present disclosure refers to Fab fragment, Fab′ fragment, F(ab′)2 fragment having antigen-binding activity, as well as Fv fragment, scFv fragment binding to human PD-1; the “antigen-binding fragment” comprises one or more CDR region(s) selected from SEQ ID NO: 1 to SEQ ID NO: 6 of the antibody described in the present disclosure. Fv fragment is a minimum antibody fragment carrying all antigen-binding sites, it comprises antibody heavy chain variable region and light chain variable region, but without constant region. Generally, Fv antibody further comprises a polypeptide linker between the VH and VL domains, and is capable of forming a structure necessary for antigen binding. Also, different linkers can be used to connect the variable regions of two antibodies to form a polypeptide chain, namely single chain antibody or single chain Fv (sFv). The term “binding to PD-1” in the present disclosure refers to the ability to interact with human PD-1. The term “antigen-binding site” in the present disclosure refers to discrete three-dimensional sites on the antigen that is recognized by the antibody or antigen-binding fragment of the present disclosure.

The “antigen-binding fragment” or “functional fragment” of the TIM-3 antibody described in the present disclosure refers to one or more fragment(s) of the antibody that retain(s) the ability to specifically bind to an antigen (for example, TIM-3). It has been shown that fragments of full-length antibody can be used to perform the antigen-binding function of antibody. Examples of the binding fragment contained in the term “antigen-binding fragment” of the antibody include (i) Fab fragment, a monovalent fragment composed of VL, VH, CL and CHI domains; (ii) F(ab′)2 fragment, a bivalent fragment including two Fab fragments connected by a disulfide bridge on the hinge region, (iii) Fd fragment composed of VH and CH1 domains; (iv) Fv fragment composed of VH and VL domains from one arm of an antibody; (v) single domain or dAb fragment (Ward et al., (1989) Nature 341: 544-546), which is composed of VH domain; and (vi) isolated complementary determining region (CDR) or (vii) combination of two or more isolated CDRs, optionally connected by synthetic linkers. In addition, although the two domains VL and VH of the Fv fragment are encoded by separate genes, recombination methods can be used to connect them through a synthetic linker so that a single protein chain can be produced in which the VL and VH regions are matched with each other to form a monovalent molecule (referred to as single chain Fv (scFv); see, for example, Bird et al. (1988) Science 242: 423-426; and Huston et al. (1988) Proc. Natl. Acad. Sci USA 85: 5879-5883). Such single chain antibody are also intended to be included in the term “antigen-binding fragment” of antibody. Such antibody fragments are obtained using conventional techniques known to those skilled in the art, and the fragments are screened for their function in the same manner as that for intact antibodies. The antigen binding portion can be produced by recombinant DNA technology or by enzymatic or chemical fragmentation of the intact immunoglobulin. The antibodies may be antibodies of different isotypes, for example IgG (for example, IgG1, IgG2, IgG3 or IgG4 subtypes), IgA1, IgA2, IgD, IgE or IgM antibody.

Fab is an antibody fragment that has a molecular weight of about 50,000 and has antigen-binding activity, which is obtained by treating IgG antibody molecules with the protease papain (cleaving the amino acid residue at position 224 of the H chain), wherein about half of the H chain at its N-terminal side and the entire L chain are connected together by disulfide bond.

The Fab described in the present disclosure can be produced by treating the monoclonal antibody of the present invention (that specifically recognizes human TIM-3 and binds to the amino acid sequence of the extracellular region or its three-dimensional structure) with papain. In addition, the Fab can be produced by inserting the DNA encoding the Fab of the antibody into a prokaryotic expression vector or a eukaryotic expression vector and introducing the vector into a prokaryotic or eukaryotic organism to express the Fab.

F(ab′)2 is an antibody fragment with a molecular weight of about 100,000 obtained by digesting the part downstream of the two disulfide bonds in the IgG hinge region with the pepsin enzyme, F(ab′)2 has antigen binding activity and comprises two Fab regions connected at the position of hinge.

The F(ab′)2 described in the present disclosure can be produced by treating the monoclonal antibody of the present invention (that specifically recognizes human TIM-3 and binds to the amino acid sequence of the extracellular region or its three-dimensional structure) with pepsin. In addition, the F(ab′)2 can be produced by linking Fab′ described below with a thioether bond or a disulfide bond.

Fab′ is an antibody fragment with a molecular weight of about 50,000 and having antigen-binding activity, which is obtained by cleaving the disulfide bond in the hinge region of F(ab′)2. The Fab′ of the present invention can be produced by treating the F(ab′)2 of the present invention (that specifically recognizes TIM-3 and binds to the amino acid sequence of the extracellular region or its three-dimensional structure) with a reducing agent such as dithiothreitol.

In addition, the Fab′ can be produced by inserting DNA encoding the Fab′ fragment of the antibody into a prokaryotic expression vector or a eukaryotic expression vector and introducing the vector into a prokaryotic organism or eukaryotic organism to express Fab′.

The “single chain antibody”, “single chain Fv” or “scFv” described in the present disclosure refers to a molecular in which the antibody heavy chain variable domain (or region; VH) is connected to the antibody light chain variable domain (or region; VL) with a linker. Such scFv molecules have general structure: NH₂-VL-linker-VH-COOH or NH₂-VH-linker-VL-COOH. A suitable linker in prior art consists of repeated GGGGS amino acid sequence(s) or variant thereof, for example 1-4 repeated variants can be used (Holliger et al. (1993), Proc. Natl. Acad. Sci. USA 90: 6444-6448). Other linkers that can be used in the present invention are described by Alfthan et al. (1995), Protein Eng. 8:725-731, Choi et al. (2001), Eur. J. Immunol. 31:94-106, Hu et al. (1996), Cancer Res. 56:3055-3061, Kipriyanov et al. (1999), J. Mol. Biol. 293:41-56 and Roovers et al. (2001), Cancer Immunol.

The scFv described in the present disclosure can be produced by the following steps: producing cDNA encoding VH and VL of the monoclonal antibody of the present invention (that specifically recognizes human TIM-3 and binds to the amino acid sequence of the extracellular region or its three-dimensional structure), constructing DNA encoding the scFv, inserting the DNA into a prokaryotic expression vector or a eukaryotic expression vector, and then introducing the expression vector into a prokaryote or eukaryotic organism to express the scFv.

The “effective amount” described in the present disclosure comprises an amount sufficient to improve or prevent the symptoms or conditions of the medical condition. An effective amount also refers to an amount sufficient to allow or facilitate diagnosis. The effective amount for a particular patient or veterinary subject can vary depending on factors such as the condition to be treated, the patient's general health, administration method, route and dosage, and the severity of side effects. The effective amount can be the maximum dosage or dosing schedule that avoids significant side effects or toxic effects.

The “CDR” described in the present disclosure refers to one of the six hypervariable regions within the variable domain of an antibody that mainly contribute to antigen binding. One of the most commonly used definition of the 6 CDRs is provided by Kabat E. A. et al. (1991) Sequences of proteins of immunological interest. NIH Publication 91-3242). As used herein, the Kabat definition of CDR only applies to the CDR1, CDR2, and CDR3 of the light chain variable domain (CDR L1, CDR L2, CDR L3 or L1, L2, L3), and the CDR2 and CDR3 of the heavy chain variable domain (CDR H2, CDR H3 or H2, H3).

The engineered antibody or antigen-binding fragment in the present disclosure can be prepared and purified by conventional methods. For example, the cDNA sequences encoding the heavy and light chains can be cloned and recombined into a GS expression vector. The recombinant immunoglobulin expression vector can be stably transfected into CHO cells. As a more recommended prior art, mammalian expression systems can lead to glycosylation of antibodies, especially at the highly conserved N-terminal sites in the Fc region. Stable clones are obtained by expressing antibodies that specifically bind to human TIM-3. Positive clones are expanded in the serum-free medium of the bioreactor to produce antibodies. The culture medium comprising secreted antibody can be purified by conventional techniques. For example, A- or G-Sepharose FF column with adjusted buffer can be used for purification. The non-specifically bound components are removed by washing. Then the bound antibody was eluted by pH gradient method, and the antibody fragment was detected by SDS-PAGE and collected. The antibody can be filtered and concentrated by conventional methods. Soluble mixtures and polymers can also be removed by conventional methods, such as molecular sieves and ion exchange. The resulting product needs to be frozen immediately, such as at −70° C., or lyophilized.

The “treatment” used in the present disclosure refers to administering an internal or external therapeutic agent, such as a composition containing any one of the binding compounds of the present invention, to a patient who has one or more disease symptoms, and the therapeutic agent is known to have a therapeutic effect on these symptoms.

Humans and animals have quite different tolerance to the same medicament. Generally speaking, animals are more tolerant than humans. Generally, the following ratios are used to perform conversion: the dosage for human is set as 1, 25-50 for mice and rats, 15-20 for rabbits and guinea pigs, and 5-10 for dogs and cats. In addition, human and animal surface area calculation methods can be used to perform conversion. 1) Human surface area calculation methods are generally considered, such as Xu Wen's formula (Chinese Journal of Physiology, 12, 327, 1937) and Mech-Rubner's formula. The above method can be applied to the conversion of medicament dosage between human and different kinds of animals in the present disclosure.

The “homology” used in the present disclosure refers to the sequence similarity between two polynucleotide sequences or between two polypeptides. When the positions in the two sequences to be compared are occupied by the same base or amino acid monomer subunit, for example, each position of the two DNA molecules is occupied by adenine, then the molecules are deemed as homologous at that position. The percentage of homology between two sequences is a function of the number of matching or homologous positions shared by two sequences divided by the number of positions to be compared×100. For example, in an optimal sequence alignment, when there are 6 matched or homologous positions among 10 positions in two sequences, then the two sequences are deemed as 60% homology; when there are 95 matched or homologous positions among 100 positions in two sequences, then the two sequences are deemed as 95% homology. Generally speaking, the comparison is performed when two sequences are aligned to obtain the maximum percent homology.

The “pharmaceutical composition” used in the present disclosure refers to a mixture containing one or more of the compounds described herein or physiologically/pharmaceutically acceptable salts or precursor thereof and other chemical components. For example, the other components are physiological/pharmaceutically acceptable carriers and excipients. The purpose of the pharmaceutical composition is to promote the administration to the organism, which contributes to the absorption of the active ingredients and thereby the biological activity.

Overall survival (OS) refers to the duration from random period to the time of death due to any cause. For subject who is still alive at the last follow-up, the OS is counted as censored data at the time of the last follow-up. For subject who is lost to follow-up, the OS is counted as censored data at the time of the last confirmed survival before being lost to follow-up. The OS with censored data is defined as the duration from random grouping to censoring.

Objective response rate (ORR) refers to the rate of patients whose tumors have shrunk to a certain level and maintained for a certain period of time, including CR and PR cases. The Response Evaluation Criteria in Solid Tumors (RECIST 1.1 Criteria) was used to assess the objective tumor response. Subjects must be accompanied with measurable tumor lesions at baseline, and the efficacy evaluation criteria are divided into complete remission (CR), partial remission (PR), stable disease (SD), and progressive disease (PD) according to the RECIST 1.1 criteria.

Disease Control Rate (DCR): The period starting from the time for first evaluation of the tumor as CR/PR/SD to the time for first evaluation as PD or death due to any cause.

12-month/24-month survival rate (Overall survival rate, OSR): The rate of cases that are still alive after 12-month/24-month follow-up since the first administration.

Disease Control Rate (DCR): refers to the rate of subjects with Best Overall Response (BOR) of complete remission (CR) or partial response (PR) or stable disease (SD>8 weeks).

Complete Remission (CR): All target lesions disappear, and the short diameter of each of the all pathological lymph nodes (including target and non-target nodes) must be reduced to <10 mm.

Partial Remission (PR): The sum of diameters of all target lesions is reduced by at least 30% from the baseline level.

Progressive Disease (PD): The sum of diameters of all target lesions is increased by at least 20% compared to the reference, which is the minimum value of said sum measured during the entire experimental study (the baseline measurement value is set as the reference, if it is the minimum value); In addition, the absolute value of the sum of diameters must be increased by at least 5 mm (the presence of one or more new lesions is also deemed as progressive disease).

Stable Disease (SD): The degree of reduction for the target lesion does not reach PR, and the degree of increase does not reach PD level, which is somewhere in between. The minimum value of the sum of diameters can be used as a reference during the study.

### DESCRIPTION OF THE DRAWINGS

FIG. 1: The effect of TIM-3 antibodies on human non-small cell lung cancer HCC827 mice xenograft tumor.

FIG. 2: The effect of antibodies on the relative tumor volume in mice.

### DETAILED DESCRIPTION OF THE DISCLOSURE

Hereinafter, the present disclosure is further described with reference to the examples. However, the scope of the present disclosure is not limited thereto.

### Example 1. Preparation of TIM-3 Antigen and Protein Used for Detection

1. Design and Expression of TIM-3 Antigen

UniProt Hepatitis A virus cellular receptor 2 (human HAVCR2, human TIM-3, Uniprot No: Q8TDQ0) was used as the template of TIM-3 of the present invention, the amino acid sequence of the antigen and protein used for detection in the present invention were designed, optionally different tags were fused to the TIM-3 protein, and then cloned into pHr vector (house-made) or pTargeT vector (promega, A1410), respectively. The vectors were transiently expressed in 293 cells or stably expressed in CHO-S, and then purified to obtain the encoded antigen and protein used for detection in the present invention. Unless indicated specifically, the following TIM-3 antigens refer to human TIM-3.

Fusion protein of TIM-3 extracellular region and hIgG1 Fc: TIM-3-Fc (SEQ ID NO: 1), used for immunization of mouse:

MEFGLSWLFLVAILKGVQCSEVEYRAEVGQNAYLPCFYTPAAPGNLVP
VCWGKGACPVFECGNVVLRTDERDVNYWTSRYWLNGDFRKGDVSLTIE
NVTLADSGIYCCRIQIPGIMNDEKFNLKLVIKPAKVTPAPTRQRDFTA
AFPRMLTTRGHGPAETQTLGSLPDINLTQISTLANELRDSRLANDLRD
SGATIREPKSSDKTHTCPPCPAPELLGGPSVFLFPPKPKDTLMISRTP
EVTCVVVDVSHEDPEVKFNWYVDGVEVHNAKTKPREEQYNSTYRVVSV
LTVLHQDWLNGKEYKCKVSNKALPAPIEKTISKAKGQPREPQVYTLPP
SRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK;

Note: The underlined part represents signal peptide, and the italicized part represents Fc.

TIM-3 Extracellular region with Flag and His tags: TIM-3-Flag-His (SEQ ID NO: 2), used for detection:

TIM-3-flag-His
MEFGLSWLFLVAILKGVQCSEVEYRAEVGQNAYLPCFYTPAAPGNLVPVC
WGKGACPVFECGNVVLRTDERDVNYWTSRYWLNGDFRKGDVSLTIENVTL
ADSGIYCCRIQIPGIMNDEKFNLKLVIKPAKVTPAPTRQRDFTAAFPRML
TTRGHGPAETQTLGSLPDINLTQISTLANELRDSRLANDLRDSGATIRGS
SDYKDDDDKHHHHHH;

Note: The underlined part represents signal peptide, and the italicized part represents Flag-His tag.

Full-length TIM-3: used to construct TIM-3-
overexpressing cell lines: TIM-3-full length
(SEQ ID NO: 3)
MFSHLPFDCVLLLLLLLLTRSSEVEYRAEVGQNAYLPCFYTPAAPGNLVP
VCWGKGACPVFECGNVVLRTDERDVNYWTSRYWLNGDFRKGDVSLTIENV
TLADSGIYCCRIQIPGIMNDEKFNLKLVIKPAKVTPAPTRQRDFTAAFPR
MLTTRGHGPAETQTLGSLPDINLTQISTLANELRDSRLANDLRDSGATIR

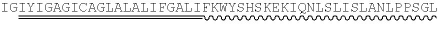

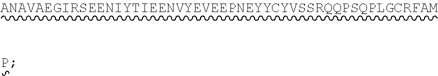

Note:
Signal peptide + extracellular region + transmembrane
region + intracellular region.

Note: Signal peptide+extracellular region+transmembrane region+intracellular region.

2. Purification of TIM-3 Related Recombinant Protein, and Purification of Hybridoma Antibodies and Recombinant Antibodies

2.1 Purification Steps of TIM-3-Flag-His Recombinant Protein:

The sample was centrifuged at high speed to remove impurities and concentrated to an appropriate volume. The NI-NTA affinity column (QIAGEN, Cat No. 30721) was equilibrated with PBS, and was washed with 2-5 times of column volume. After removing the impurities, the cell expression supernatant sample was loaded onto the column. The column was rinsed with PBS until the A280 reading dropped to the baseline. The column was rinsed with PBS to wash impurity proteins, and the target protein was collected. The target protein was eluted with washing buffer (20 mM imidazole) and elution buffer (300 mM imidazole) successively, and the elution peaks were collected.

The collected eluate was further purified by ion exchange (Hiload 16/600 Superdex 200 column). The column was equilibrated with about 2 column volumes of PBS to ensure pH 7.4. The elution buffer which has been identified to comprise the target protein was loaded after concentration, and the sample was collected, identifed by using SDS-PAGE and LC-MS, and was aliquoted for later use.

2.2 Purification of Hybridomas, Recombinant Antibodies, and Fc Fusion Proteins

The cell expression supernatant sample was centrifuged at high speed to remove impurities, the hybridoma expression supernatant was purified by Protein G column, and the recombinant antibody and Fc fusion protein expression supernatant were purified by Protein A column. The column was rinsed with PBS until the A280 reading dropped to the baseline. The target protein was eluted with 100mM acetic acid pH3.0, and neutralized with 1M Tris-HC1 pH8.0. The eluted sample was appropriately concentrated and further purified by PBS-equilibrated gel chromatography Superdex 200 (GE). The non-aggregate peaks were collected and aliquoted for later use.

### Example 2. Preparation of Anti-Human TIM-3 Monoclonal Antibody

1. Animal Immunization

Anti-human TIM-3 monoclonal antibody was produced by immunizing mice. SJL white mice, female, 6-8 weeks old (Beijing Charles River Laboratory Animal Technology Co., Ltd., animal production license number: SOCK (Beijing) 2012-0001) were used in the experiment. Feeding environment: SPF level. After the mice were purchased, they were adapted to the laboratory environment for 1 week, 12/12 hours light/dark cycle adjustment, temperature 20-25° C.; humidity 40-60%. Mice that have been adapted to the environment were immunized according to the following protocol. The antigen for immunization was the extracellular region of human TIM-3 with Fc-tag (SEQ ID NO: 1).

Immunization protocol: QuickAntibody-Mouse5W (KX0210041) was used to immunize mice. The ratio of antigen to adjuvant is 1:1, 10 μg/mouse/time (first immunization/booster immunization). The antigen and adjuvant were quickly and thoroughly mixed and then inoculated. The inoculation period involved an interval of 21 days between the first and second immunizations, and an interval of 14 days between later immunizations. Blood was taken 7 days after each immunization, and the antibody titer in the mouse serum was determined by ELISA. The mice with high antibody titer in serum and with its titer reaching to the plateau were selected for splenocyte fusion. Three days before the fusion of splenocytes, the booster immunization was performed, and antigen solution prepared by physiological saline was injected at 20 μg/mouse by intraperitoneally (IP).

2. Splenocyte Fusion

Optimized PEG-mediated fusion steps were used to fuse splenic lymphocytes with myeloma cells Sp2/0 cells (ATCC® CRL-8287™) to obtain hybridoma cells. The fused hybridoma cells were re-suspended in complete medium (DMEM medium comprising 20% FBS, 1×HAT, 1×OPI) at a density of 4-5 E5/ml, and were seeded onto a 96-well plate at 100 μl/well, and incubated at 37° C. in 5% CO₂ for 3-4 days, and then HAT complete medium was added at 100 μl/l well to further cultivate the cells for 3-4 days until pinpoint-like clones were formed. The supernatant was removed, 200 μl/well of HT complete medium (RPMI-1640 medium comprising 20% FBS, 1×HT and 1×OPI) was added, and incubated at 37° C. in 5% CO₂ for 3 days, and then ELISA detection was performed.

3. Screening of Hybridoma Cell

According to the growth density of hybridoma cells, the hybridoma culture supernatant was detected by binding ELISA method (see Example 4, Test Example 1). TIM-3 overexpressing cell binding experiment was performed using cell supernatant in positive wells which were identified in the binding ELISA method (see Example 4, Test Example 2). The cells in wells that are positive for both protein-binding and cell-binding should be expanded in time for cryopreservation, and subcloned two to three times until single cell clone can be obtained.

TIM-3 binding ELISA and cell binding experiments were required for each subcloning of cells. The hybridoma clones were screened through the above experiments, and the secreted antibodies mAb-1701 and mAb-1799 were obtained. The antibodies were further prepared by the serum-free cell culture method. The antibodies were purified according to the purification example, and were provided for use in the test examples.

4. Sequencing of Hybridoma Positive Clones

The process for cloning the sequences from the positive hybridoma was as follows. The hybridoma cells at logarithmic growth phase were collected, RNA was extracted by Trizol (Invitrogen, Cat No. 15596-018) according to the kit instructions, and PrimeScript™ Reverse Transcriptase kit was used for reverse transcription (Takara, Cat No. 2680A). The cDNA obtained by reverse transcription was amplified by PCR using mouse Ig-Primer Set (Novagen, TB326 Rev. B 0503) and was delivered to company for sequencing. The amino acid sequences corresponding to the DNA sequences for heavy chain and light chain variable region(s) of mAb-1701 and mAb-1799 were obtained:

mAb-1701 heavy chain variable region
(SEQ ID NO: 4)
EVQLQQSGPELVKPGASVKISCKASGYTFTDYYMNWVKQSHGKSLEWIA
DIIPNNGGSKYNQKFKDKATLTVDKSSSTAYMELRSLTSEDSAVYYCAT
WGYGSSYRWFDYWGQGTLVSVSA;
mAb-1701 light chain variable region
(SEQ ID NO: 5)
DIQMTQSPASQSASLGESVTITCLASQPIGIWLAWYQQKPGKSPQLLIY
AATSLADGVPSRFSGSGSGTKFSFKISSLQAEDFVSYYCQQLYSSPWTFG
GGTKLEIK;
mAb-1799 heavy chain variable region
(SEQ ID NO: 6)
EVKLVESEGGLVQPGSSMKLSCTASGFTFSDYYMAWVRQVPEKGLEWVA
NINYDGSSTYYLDSLKSRFIISRDNAKNILYLQMNSLKSDDTATYYCAR
DVGYYGGNYGFAYWGQGTLVTVSA;
mAb-1799 light chain variable region
(SEQ ID NO: 7)
DIQMTQSPASLSASVGETVTITCRASDNIYSYLAWYQQKQGKSPQLLVY
NAKTLAEGVPSRFSGSGSGTQFSLKINSLQPEDFGSYYCQQHYGSPLTFG
AGTKLELK.

Wherein, the CDR sequences in the light and heavy chains of each antibody are shown in Table 1.

TABLE 1
Sequences of CDRs of each heavy
chain and light chain

| Ab | Heavy chain | | Light chain | |
|---|---|---|---|---|
| 1701 | HCDR1 | DYYMN | LCDR1 | LASQPIGIWLA |
| | | SEQ ID | | SEQ ID |
| | | NO: 8 | | NO: 11 |
| | HCDR2 | DIIPNNGGS | LCDR2 | AATSLAD |
| | | KYNQKFKD | | SEQ ID |
| | | SEQ ID | | NO: 12 |
| | | NO: 9 | | |
| | HCDR3 | WGYGSSYRWFDY | LCDR3 | QQLYSSPWT |
| | | SEQ ID | | SEQ ID |
| | | NO: 10 | | NO: 13 |
| 1799 | HCDR1 | DYYMA | LCDR1 | RASDNIYSYLA |
| | | SEQ ID | | SEQ ID |
| | | NO: 14 | | NO: 17 |
| | HCDR2 | NINYDGSST | LCDR2 | NAKTLAE |
| | | YYLDSLKS | | SEQ ID |
| | | SEQ ID | | NO: 18 |
| | | NO: 15 | | |
| | HCDR3 | DVGYYGG | LCDR3 | QQHYGSPLT |
| | | NYGFAY | | SEQ ID |
| | | SEQ ID | | NO: 19 |
| | | NO: 16 | | |

### Example 3. Humanization of Anti-Human TIM-3 Murine Hybridoma Monoclonal Antibody

1. Humanization of Anti-TIM-3 Antibody mAb-1701

By aligning against IMGT germline gene database of human antibody heavy and light chain variable region by MOE software, the heavy chain and light chain variable region germline genes with high homology to mAb-1701 antibody were selected as templates, and the CDRs of murine antibody were respectively grafted onto the corresponding human template to form the variable region in the order of FR1-CDR1-FR2-CDR2-FR3-CDR3-FR4. The amino acid residues are identified and annotated by Kabat numbering system.

1.1 Humanized Framework Selection for Hybridoma Clone mAb-1701

The light chain templates for humanization of the murine antibody mAb-1701 were IGKV1-33*01 and hjk4.1, and the heavy chain templates for humanization were IGHV1-18*01 and hjh4.1. The humanized variable region sequences are as follows:

h1701VH-CDR graft
(SEQ ID NO: 20)
QVQLVQSGAEVKKPGASVKVSCKASGYTFTDYYMNWVRQAPGQGLEWM
GDIIPNNGGSKYNQKFKDRVTMTTDTSTSTAYMELRSLRSDDTAVYYC
ARWGYGSSYRWFDYWGQGTLVTVSS;
h1701VL-CDR graft
(SEQ ID NO: 21)
DIQMTQSPSSLSASVGDRVTITCLASQPIGIWLAWYQQKPGKAPKLLI
YAATSLADGVPSRFSGSGSGTDFTFTISSLQPEDIATYYCQQLYSSPW
TFGGGTKVEIK;

Note: The order is FR1-CDR1-FR2-CDR2-FR3-CDR3-FR4, the italics in the sequence represent FR sequence, and the underlined part represents CDR sequences.

1.2 Template Selection and Back-Mutation(s) Design for h1701

The specific mutation design is shown in Table 2 below:

TABLE 2
Template selection and back-mutation(s) design for h1701

| h1701_VL | | h1701 VH | |
|---|---|---|---|
| h1701_VL.1 | Grafted | h1701_VH.1 | Grafted |
| h1701_VL.1A | A43S | h1701_VH.1A | M48I |
| | | h1701_VH.1B | R98T |
| | | h1701_VH.1C | M48I, R98T |
| | | h1701_VH.1D | M48I, R98T, R38K, D89E |
| | | h1701_VH.1E | M48I, R98T, G49A, V68A, |
| | | | M70L |
| | | h1701_VH.1F | M48I, R98T, G49A, V68A, |
| | | | M70L, R38K, D89E |

Note: For example, A43S means that A at position 43 is mutated back to S, according to the Kabat numbering system. “Grafted” represents the sequence of murine antibody CDRs implanted into human germline FR region.

TABLE 3
Combination of h1701 humanized antibody heavy chain variable
region and light chain variable region sequences

| | h1701_VL.1 | h1701_VL.1A |
|---|---|---|
| h1701_VH.1 | h1701-005 | h1701-006 |
| h1701_VH.1A | h1701-007 | h1701-008 |
| h1701_VH.1B | h1701-009 | h1701-010 |
| h1701_VH.1C | h1701-011 | h1701-012 |
| h1701_VH.1D | h1701-013 | h1701-014 |
| h1701_VH.1E | h1701-015 | h1701-016 |
| h1701_VH.1F | h1701-017 | h1701-018 |

Note: This table shows the sequences resulted from various combinations of the mutations. As indicated by h1701-007, the humanized murine antibody h1701-007 has two mutants (light chain h1701_VL.1A and heavy chain h1701_VH.1A). Others can be indicated in similar way.

The particular sequence of humanized 1701 is as follows:

>h1701_VH.1
(the same as h1701VH-CDR graft, SEQ ID NO: 22)
QVQLVQSGAEVKKPGASVKVSCKASGYTFTDYYMNWVRQAPGQGLEWMGD
IIPNNGGSKYNQKFKDRVTMTTDTSTSTAYMELRSLRSDDTAVYYCARWG
YGSSYRWFDYWGQGTLVTVSS;
>h1701h1701_VH.1A
(SEQ ID NO: 23)
QVQLVQSGAEVKKPGASVKVSCKASGYTFTDYYMNWVRQAPGQGLEWIGD
IIPNNGGSKYNQKFKDRVTMTTDTSTSTAYMELRSLRSDDTAVYYCARWG
YGSSYRWFDYWGQGTLVTVSS;
>h1701_VH.1B
(SEQ ID NO: 24)
QVQLVQSGAEVKKPGASVKVSCKASGYTFTDYYMNWVRQAPGQGLEWMGD
IIPNNGGSKYNQKFKDRVTMTTDTSTSTAYMELRSLRSDDTAVYYCATWG
YGSSYRWFDYWGQGTLVTVSS;
>h1701_VH.1C
(SEQ ID NO: 25)
QVQLVQSGAEVKKPGASVKVSCKASGYTFTDYYMNWVRQAPGQGLEWIGD
IIPNNGGSKYNQKFKDRVTMTTDTSTSTAYMELRSLRSDDTAVYYCATWG
YGSSYRWFDYWGQGTLVTVSS;
>h1701_VH.1D
(SEQ ID NO: 26)
QVQLVQSGAEVKKPGASVKVSCKASGYTFTDYYMNWVKQAPGQGLEWIGD
IIPNNGGSKYNQKFKDRVTMTTDTSTSTAYMELRSLRSEDTAVYYCATWG
YGSSYRWFDYWGQGTLVTVSS;
>h1701_VH.1E
(SEQ ID NO: 27)
QVQLVQSGAEVKKPGASVKVSCKASGYTFTDYYMNWVRQAPGQGLEWIAD
IIPNNGGSKYNQKFKDRATLTTDTSTSTAYMELRSLRSDDTAVYYCATWG
YGSSYRWFDYWGQGTLVTVSS;
>h1701_VH.1F
(SEQ ID NO: 28)
QVQLVQSGAEVKKPGASVKVSCKASGYTFTDYYMNWVKQAPGQGLEWIAD
IIPNNGGSKYNQKFKDRATLTTDTSTSTAYMELRSLRSEDTAVYYCATWG
YGSSYRWFDYWGQGTLVTVSS;
>h1701_VL.1
(the same as h1701VL-CDR graft, SEQ ID NO: 29)
DIQMTQSPSSLSASVGDRVTITCLASQPIGIWLAWYQQKPGKAPKLLIYA
ATSLADGVPSRFSGSGSGTDFTFTISSLQPEDIATYYCQQLYSSPWTFGG
GTKVEIK;
>h1701_VL.1A
(SEQ ID NO: 30)
DIQMTQSPSSLSASVGDRVTITCLASQPIGIWLAWYQQKPGKSPKLLIYA
ATSLADGVPSRFSGSGSGTDFTFTISSLQPEDIATYYCQQLYSSPWTFGG
GTKVEIK.

2. Humanization of Anti-TIM-3 Antibody mAb-1799

By aligning against IMGT germline gene database of human antibody heavy and light chain variable region by MOE software, the heavy chain and light chain variable region germline genes with high homology to mAb-1799 antibody were selected as templates, and the CDRs of murine antibody were respectively grafted onto the corresponding human template to form the variable region in the order of FR1-CDR1-FR2-CDR2-FR3-CDR3-FR4. The amino acid residues are identified and annotated by Kabat numbering system.

2.1 Humanized Framework Selection for Hybridoma Clone 1799

The light chain templates for humanization of the murine antibody 1799 were IGKV1-39*01 and hjk2.1, and the heavy chain templates for humanization were IGHV3-7*01 and hjh4.1. The humanized variable region sequences are as follows:

h1799VH-CDR graft
(SEQ ID NO: 31)
EVQLVESGGGLVQPGGSLRLSCAASGFTFSDYYMAWVRQAPGKGLEWV
ANINYDGSSTYYLDSLKSRFTISRDNAKNSLYLQMNSLRAEDTAVYYC
ARDVGYYGGNYGFAYWGQGTLVTVSS;
h1799VL-CDR graft
(SEQ ID NO: 32)
DIQMTQSPSSLSASVGDRVTITCRASDNIYSYLAWYQQKPGKAPKLLI
YNAKTLAEGVPSRFSGSGSGTDFTLTISSLQPEDFATYYCQQHYGSPL
TFGQGTKLEIK;

Note: The order is FR1-CDR1-FR2-CDR2-FR3-CDR3-FR4, the italics in the sequence represent FR sequence, and the underlined part represents CDR sequences.

2.2 Template Selection and Back-Mutation(s) Design of Hybridoma Clone 1799, see Table 4 Below:

TABLE 4
Template selection and back-mutation(s) design of h1799

| h1799_VL | | h1799_VH | |
|---|---|---|---|
| h1799_VL.1 | Grafted | h1799_VH.1 | Grafted |
| h1799_VL.1A | I48V | h1799_VH.1A | Q3K |
| h1799_VL.1B | I48V, K45Q | h1799_VH.1B | Q3K, R87K |
| h1799_VL.1C | I48V, K45Q, A43S | | |
| h1799_VL.1D | I48V, K45Q, A43S, | | |
| | T85S | | |

Note: For example, I48V means that I at position 48 is mutated back to V, according to the

Kabat numbering system. “Grafted” represents the sequence of murine antibody CDRs implanted into human germline FR region.

TABLE 5
Combination of humanized antibody heavy chain variable region and
light chain variable region sequences, for murine antibody 1799

| | h1799_VL.1 | h1799_VL.1A | h1799_VL.1B | h1799_VL.1C | h1799_VL.1D |
|---|---|---|---|---|---|
| h1799_VH.1 | h1799-005 | h1799-006 | h1799-007 | h1799-008 | h1799-009 |
| h1799_VH.1A | h1799-010 | h1799-011 | h1799-012 | h1799-013 | h1799-014 |
| h1799_VH.1B | h1799-015 | h1799-016 | h1799-017 | h1799-018 | h1799-019 |

Note: This table shows the sequences resulted from various combinations of the mutations. As indicated by h1799-005, the humanized murine antibody h1799-005 has two mutants (light chain h1799_VL.1A and heavy chain h1799_VH.1). Others can be indicated in similar way.

The particular sequence of the humanized 1799 is as follows:

>h1799_VH.1
(the same as h1799VH-CDR graft, SEQ ID NO: 33)
EVQLVESGGGLVQPGGSLRLSCAASGFTFSDYYMAWVRQAPGKGLEWVAN
INYDGSSTYYLDSLKSRFTISRDNAKNSLYLQMNSLRAEDTAVYYCARDV
GYYGGNYGFAYWGQGTLVTVSS;
>h1799_VH.1A
(SEQ ID NO: 34)
EVKLVESGGGLVQPGGSLRLSCAASGFTFSDYYMAWVRQAPGKGLEWVAN
INYDGSSTYYLDSLKSRFTISRDNAKNSLYLQMNSLRAEDTAVYYCARDV
GYYGGNYGFAYWGQGTLVTVSS;
>h1799_VH.1B
(SEQ ID NO: 35)
EVKLVESGGGLVQPGGSLRLSCAASGFTFSDYYMAWVRQAPGKGLEWVAN
INYDGSSTYYLDSLKSRFTISRDNAKNSLYLQMNSLKAEDTAVYYCARDV
GYYGGNYGFAYWGQGTLVTVSS;
>h1799_VL.1
(the same as h1799VL-CDR graft, SEQ ID NO: 36)
DIQMTQSPSSLSASVGDRVTITCRASDNIYSYLAWYQQKPGKAPKLLIYN
AKTLAEGVPSRFSGSGSGTDFTLTISSLQPEDFATYYCQQHYGSPLTFGQ
GTKLEIK;
>h1799_VL.1A
(SEQ ID NO: 37)
DIQMTQSPSSLSASVGDRVTITCRASDNIYSYLAWYQQKPGKAPKLLVYN
AKTLAEGVPSRFSGSGSGTDFTLTISSLQPEDFATYYCQQHYGSPLTFGQ
GTKLEIK;
>h1799_VL.1B
(SEQ ID NO: 38)
DIQMTQSPSSLSASVGDRVTITCRASDNIYSYLAWYQQKPGKAPQLLVYN
AKTLAEGVPSRFSGSGSGTDFTLTISSLQPEDFATYYCQQHYGSPLTFGQ
GTKLEIK;
>h1799_VL.1C
(SEQ ID NO: 39)
DIQMTQSPSSLSASVGDRVTITCRASDNIYSYLAWYQQKPGKSPQLLVYN
AKTLAEGVPSRFSGSGSGTDFTLTISSLQPEDFATYYCQQHYGSPLTFGQ
GTKLEIK;
>h1799_VL.1D
(SEQ ID NO: 40)
DIQMTQSPSSLSASVGDRVTITCRASDNIYSYLAWYQQKPGKSPQLLVYN
AKTLAEGVPSRFSGSGSGTDFTLTISSLQPEDFASYYCQQHYGSPLTFGQ
GTKLEIK.

### Example 4. Preparation and Effect Test of Recombinant Chimeric Antibody and Humanized Antibody

For the antibodies, the constant regions of human heavy chain IgG4/light chain kappa were combined with each of the corresponding variable regions, and S228P mutation was introduced in the Fc section to increase the stability of the IgG4 antibody. Other mutations known in the art can also be used to improve its performance.

The sequence of the heavy chain constant region is
shown in SEQ ID NO: 41:
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK;
The sequence of the light chain constant region is
as shown in SEQ ID NO: 42:
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC.

1. Molecular Cloning of Recombinant Chimeric Antibodies

The positive antibody molecules obtained from hybridoma screening were sequenced to obtain the sequence of variable region coding gene. The forward and reverse primers were designed based on the sequence obtained by sequencing, and the sequenced gene was served as template; various antibody VH/VK gene fragments were constructed by PCR, and then homologously recombined with expression vector pHr (with signal peptide and hIgG4/hkappa constant region gene (CH1-FC/CL) fragment), and recombinant chimeric antibody full-length expression plasmids VH-CH1-FC-pHr/VL-CL -pHr were constructed for the two chimeric antibodies Ch1701 and Ch1799.

2. Molecular Cloning of Humanized Antibodies

The antibody sequences after humanization design were subjected to codon optimization to obtain the coding gene sequence having human codon preference, primers were designed to construct various antibody VH/VK gene fragments by PCR, and then the fragments were homologously recombined with expression vector pHr (with signal peptide and hIgG4/hkappa constant region gene (CH1-FC/CL) fragment) to construct humanized antibody full-length expression plasmid VH-CH1-FC-pHrNL-CL-pHr.

3. Expression and Purification of Recombinant Chimeric Antibodies and Humanized Antibodies

The plasmids separately expressing antibody light chain and heavy chain were transfected into HEK293E cells at a ratio of 1:1.2; the expression supernatant was collected 6 days later and centrifuged at high speed to remove impurities; and was purified with Protein A column. The column was rinsed with PBS until the A280 reading dropped to the baseline. The target protein was eluted with acidic elution solution, pH 3.0-pH 3.5, and was neutralized with 1M Tris-HC1 pH 8.0-9.0. The eluted sample was appropriately concentrated and further purified by PBS-equilibrated gel chromatography Superdex 200 (GE). The aggregate peaks were removed, and the monomer peaks were collected and aliquoted for later use.

### Example 5. Site-Directed Mutation of h1701 Antibody

Deamidation modification is a common chemical modification in antibodies that may affect the stability at later stage. Particularly, some amino acids in the CDR region(s) are highly deamidated, oxidized or isomerized; generally such mutations should be avoided or reduced as much as possible. According to accelerated stability experiments and computer-simulated antibody structure as well as hotspot prediction, the NNG in the heavy chain CDR2 of the h1701 antibody is the site susceptible to deamidation. The NNG described above are located at positions 54-56 in the heavy chain variable region of h1701 antibody respectively. According to properties of amino acids and technology for computer-simulated antibody structure, the amino acids at the above positions can be replaced with any amino acid. Preferably, the CDR2 mutant of h1701 is shown as: DIIPX₁X₂X₃GSKYNQKFKD (SEQ ID NO: 43), where X₁, X₂ and X₃ are amino acid residues at positions 54-56 in h1701 antibody heavy chain variable region; X₁ is selected from the group consisting of Asn, Leu, Val, Met and Glu; X₂ is selected from the group consisting of Asn, Glu, Met, His, Lys, Leu, Ala and Val; and X₃ is selected from the group consisting of Gly and Ala.

Further, the CDR2 comprising mutations at positions 54-56 described above can combine with the FR region comprising different back-mutation(s) to form the following heavy chain variable regions:

>h1701_VH.1-CDR2 mutant
(SEQ ID NO: 44)
QVQLVQSGAEVKKPGASVKVSCKASGYTFTDYYMNWVRQAPGQGLEWMGD
IIPX₁X₂X₃GSKYNQKFKDRVTMTTDTSTSTAYMELRSLRSDDTAVYYCA
RWGYGSSYRWFDYWGQGTLVTVSS;
>h1701_VH.1A-CDR2 mutant
(SEQ ID NO: 45)
QVQLVQSGAEVKKPGASVKVSCKASGYTFTDYYMNWVRQAPGQGLEWIGD
IIPX₁X₂X₃GSKYNQKFKDRVTMTTDTSTSTAYMELRSLRSDDTAVYYCA
RWGYGSSYRWFDYWGQGTLVTVSS;
>h1701_VH.1B-CDR2 mutant
(SEQ ID NO: 46)
QVQLVQSGAEVKKPGASVKVSCKASGYTFTDYYMNWVRQAPGQGLEWMGD
IIPX₁X₂X₃GSKYNQKFKDRVTMTTDTSTSTAYMELRSLRSDDTAVYYCA
TWGYGSSYRWFDYWGQGTLVTVSS;
>h1701_VH.1C-CDR2 mutant
(SEQ ID NO: 47)
QVQLVQSGAEVKKPGASVKVSCKASGYTFTDYYMNWVRQAPGQGLEWIGD
IIPX₁X₂X₃GSKYNQKFKDRVTMTTDTSTSTAYMELRSLRSDDTAVYYCA
TWGYGSSYRWFDYWGQGTLVTVSS;
>h1701_VH.1D-CDR2 mutant
(SEQ ID NO: 48)
QVQLVQSGAEVKKPGASVKVSCKASGYTFTDYYMNWVKQAPGQGLEWIGD
IIPX₁X₂X₃GSKYNQKFKDRVTMTTDTSTSTAYMELRSLRSEDTAVYYCA
TWGYGSSYRWFDYWGQGTLVTVSS;
>h1701_VH.1E-CDR2 mutant
(SEQ ID NO: 49)
QVQLVQSGAEVKKPGASVKVSCKASGYTFTDYYMNWVRQAPGQGLEWIAD
IIPX₁X₂X₃GSKYNQKFKDRATLTTDTSTSTAYMELRSLRSDDTAVYYCA
TWGYGSSYRWFDYWGQGTLVTVSS;
>h1701_VH.1F-CDR2 mutant
(SEQ ID NO: 50)
QVQLVQSGAEVKKPGASVKVSCKASGYTFTDYYMNWVKQAPGQGLEWIAD
IIPX₁X₂X₃GSKYNQKFKDRATLTTDTSTSTAYMELRSLRSEDTAVYYCA
TWGYGSSYRWFDYWGQGTLVTVSS.

Exemplary sequences related to the HCDR2 mutants of h1701 and the humanized sequence h1701_VH.1B-CDR2 mutant (SEQ ID NO: 46) comprising the corresponding CDR2 mutant are shown in the following mutants and Table 6.

As an example, the NNG in HCDR2 of h1701-009 was designed to be mutated as NLG, NVG, NNA, NMA, NEA, NHA, NMG, NEG, NKG, NAG or NHG (the sequences of the above heavy chain variable region CDR2 amino acid mutants are as shown in SEQ ID NOs: 51-61 respectively). The expression plasmid construction and 293E expression were carried out by method of molecular cloning, and the mutant antibodies were purified and then further tested for the affinity and stability.

The affinity detection results of the exemplary variants are shown in Test Examples 1 and 3 respectively.

A series of amino acid mutations were performed on h1701-009, the particularly related sequences include but not limited to those described in Table 6. The particular results of chemical stability test are shown in Test Example 9:

TABLE 6
Sequences of heavy chain variable region mutants
of h1701-009 comprising anti-deamidation modification

| Heavy chain | SEQ ID NO. | |
|---|---|---|
| variable region | for VH | Corresponding HCDR2 sequence |
| h1701-009 | SEQ ID NO: 24 | DIIPNNGGSKYNQKFKD (SEQ ID NO: 9) |
| h1701-009NLG | SEQ ID NO: 51 | DIIPNLGGSKYNQKFKD (SEQ ID NO: 62) |
| h1701-009NVG | SEQ ID NO: 52 | DIIPNVGGSKYNQKFKD (SEQ ID NO: 63) |
| h1701-009NNA | SEQ ID NO: 53 | DIIPNNAGSKYNQKFKD (SEQ ID NO: 64) |
| h1701-009NMA | SEQ ID NO: 54 | DIIPNMAGSKYNQKFKD (SEQ ID NO: 65) |
| h1701-009NEA | SEQ ID NO: 55 | DIIPNEAGSKYNQKFKD (SEQ ID NO: 66) |
| h1701-009NHA | SEQ ID NO: 56 | DIIPNHAGSKYNQKFKD (SEQ ID NO: 67) |
| h1701-009NMG | SEQ ID NO: 57 | DIIPNMGGSKYNQKFKD (SEQ ID NO: 68) |
| h1701-009NEG | SEQ ID NO: 58 | DIIPNEGGSKYNQKFKD (SEQ ID NO: 69) |
| h1701-009NKG | SEQ ID NO: 59 | DIIPNKGGSKYNQKFKD (SEQ ID NO: 70) |
| h1701-009NAG | SEQ ID NO: 60 | DIIPNAGGSKYNQKFKD (SEQ ID NO: 71) |
| h1701-009NHG | SEQ ID NO: 61 | DIIPNHGGSKYNQKFKD (SEQ ID NO: 72) |

### Test Example 1: Evaluation and Comparison of the Therapeutic Effect of TIM-3 Antibodies on Human Non-Small Cell Lung Cancer Subcutaneous Xenograft in HCC827 Mice

Laboratory Animals and Breeding Conditions

NOG female mice were purchased from Beijing Charles River Laboratory Animal Technology Co., Ltd., (Beijing China, Certificate number 11400700200456, license SCXK (Beijing) 2016-0006), 4-6 week-old at the time of purchase, weighed about 18 g, kept at 5 mice/cage, with 12/12 hours light/dark cycle adjustment, constant temperature of 23±1° C., humidity of 50% to 60%, and food and water ad libitum.

Antibodies to be tested:

C25-hIgG4 (WTRC25, U.S. Pat. No. 6,114,143), at a concentration of 5.39 mg/ml, and deliver quantity was 37.73 mg.

h1799-005, at concentration of 12.00 mg/ml, and deliver quantity was 27 mg.

MBG-453 (Novartis AG), at a concentration of 5.44 mg/ml, and deliver quantity was 25 mg.

h1701-009NLG, at a concentration of 6.30 mg/ml, and deliver quantity was 24 mg.

Preparation method: the antibodies above were diluted to a concentration of 2 mg/ml with PBS using pyrogen-free pipette tip under aseptic condition, divided into total of 10 tubes, 1.2 ml/tube, stored at 4° C.; 1 tube was taken out for each injection.

PBMCs Extraction

The PBMCs used in this experiment were extracted from fresh blood of two volunteers. The extraction method was as follows:

a) The venous blood was treated with heparin to prevent agglutination, and mixed with equal volume of PBS comprising 2% FBS;

b) 15 ml of separation solution 1077 was aseptically transferred into a 50 ml separation tube (inverting the tube gently to fully mix 1077 in advance);

c) 25 ml of diluted blood was carefully added to 1077 in a centrifuge tube (at room temperature, added slowly to form an obvious layer between blood and 1077; without mixing the diluted blood with 1077);

d) The sample was centrifuged at 1200 g for 10 minutes at room temperature. Red blood cells and multi-nucleated white blood cells were precipitated by centrifugation, and meanwhile a layer of mononuclear lymphocytes was formed above 1077. The plasma 4-6 cm above the lymphocytes was aspirated;

e) The lymphocyte layer and half of 1077 below the lymphocyte layer were aspirated and transferred to another centrifuge tube. An equal volume of PBS was added and centrifuged at 300g for 8 minutes at room temperature;

f) The cells were washed with PBS or RPMI-1640 medium, and re-suspended with RPMI-1640 medium comprising serum.

Experimental Steps:

200 μl of HCC827 cells (1×10\hat{ }7cells/mouse) (comprising 50% matrigel) were inoculated subcutaneously at right ribs of NOG mice. 16 days later, animals carrying too large or too small tumors were excluded, mice with the average tumor volume of about 215 mm\hat{ }3 were randomly divided into 4 groups: irrelevant antibody C25 IgG4 10 mpk, MBG-453 10 mpk, h1799-005 10 mpk and h1701-009NLG 10 mpk, 10 mice in each group (Day0); during the experiment, one animal in group #60-008L 10 mpk exhibited persistent weight loss after injection of PBMCs and died on Day19 (suspected suffering from GVHD). In fact, 9 animals were included. PBMCs freshly extracted from two volunteers were mixed at a ratio of 1:1, and the mixture was injected intraperitoneally into NOG mice at 5×10\hat{ }6 cells/100 μl, and each of the antibodies was also injected intraperitoneally, twice per week for 7 times in total (Table 1); the tumor volume and animal weight were monitored twice per week, the data was recorded. At the end of the experiment, the animals were euthanized, and the tumor was taken and weighed.

Data Processing

Plotting and statistical analysis of all data were performed by using Excel and GraphPad Prism 5 software.

The tumor volume (V) was calculated according to the formula: V=½×a×b², wherein a and b represent length and width, respectively.

Relative tumor proliferation rate T/C (%)=(T−T₀)/(C−C₀)×100, wherein T and C represent the tumor volume of the treatment group and the control group at the end of the experiment; T₀ and C₀ represent the tumor volume at the beginning of the experiment.

Tumor inhibition rate TGI (%)=1−T/C (%).

TABLE 7
The therapeutic effect of TIM-3 antibodies on human
non-small cell lung cancer xenograft in HCC827 mice

| | | | | D 0 | D 21 | |
|---|---|---|---|---|---|---|
| | Numbers of | administration | administration | Mean ± SEM | Mean ± SEM | TGI |
| Group | animals^{a} | cycle | route | (mm³) | (mm³) | (%) |
| C25 IgG4 | 10(10) | BIW × 7 | I.P. | 215.6 ± 12.1 | 577.1 ± 82.9 | |
| MBG-453 | 10(10) | BIW × 7 | I.P. | 215.0 ± 11.9 | 294.0 ± 77.1* | 78.15 |
| h1799-005 | 10(10) | BIW × 7 | I.P. | 215.1 ± 12.6 | 139.7 ± 14.3*** | 120.86 |
| h1701-009 | 9(10) | BIW × 7 | I.P. | 210.5 ± 15.5 | 264.7 ± 89.2* | 85.01 |
| NLG | | | | | | |

D 0: the time for the first administration;
^{a}actual numbers (grouping numbers)
**p < 0.01,
***p < 0.001 v.s. C25 IgG4-10 mpk by two-way ANOVA, Bonferroni's post-hoc test,
i.p.: intraperitoneal injection.

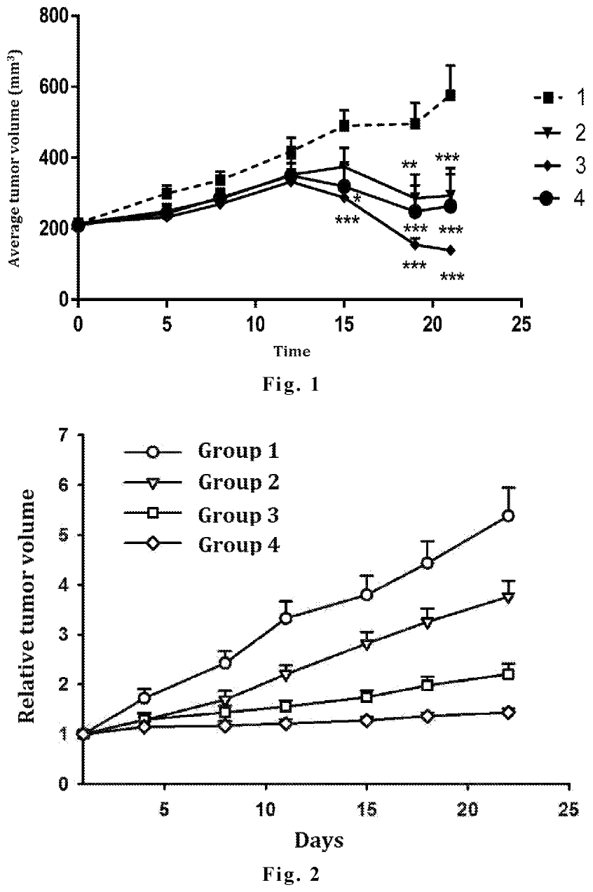

Experimental results show that: Three TIM-3 antibodies MBG-453 (10 mpk, LP., BIW×7), h1799-005 (10 mpk, LP., BIW×7) and h1701-009NLG (10 mpk, I.P., BIW×7) can significantly inhibit the growth of human non-small cell lung cancer subcutaneous xenograft in HCC827 mice. On Day21 (the last measurement), the average volume of tumor in order from small to large is h1799-005 (10 mpk, LP., BIW×7), h1701-009NLG (10 mpk, I.P., BIW×7) and MBG-453 (10 mpk, I.P., BIW×7), respectively; and the tumor inhibition rates were 120.86% (p<0.001), 85.01% (p<0.05) and 78.15% (p<0.05) respectively (see Table 7 and FIG. 1).

The tumor weights in vitro show tendency consistent with that observed for tumor volumes. The tumor weights of the three TIM-3 antibody groups were all significantly smaller than that of the irrelevant antibody C25 IgG4 (10 mpk, LP., BIW×7), and h1799-005 (10 mpk, I.P., BIW×7), h1701-009NLG (10 mpk, I.P., BIW×7) and MBG-453 (10 mpk, LP., BIW×7) exhibit the smallest, medium and the largest weights, respectively. All groups exhibited significant difference from C25 IgG4 (10 mpk, LP., BIW×7), p<0.001, p<0.05, and p<0.05 respectively.

Tumor-bearing mice were well tolerant to all TIM-3 antibodies, and only showed a slight change in the body weight during the whole administration process, no medicament-induced symptoms, such as obvious weight loss, were observed, except for one animal in h1701-009NLG (10 mpk, I.P., BIW×7) group, which exhibited persistent weight loss after injection of PBMCs and was found dead on Dayl9 (the animal's abdomen was black when it was found dead, and the skin got rotten when being touched with tweezers, with obvious foul smell; the postmortem interval was estimated to be longer than 8 hours; considering the persistent weight loss before death, it was suspected suffering from GVHD due to being intolerant to the xenograft after transplantation of human PBMCs).

### Test Example 2: Evaluation and Comparison of the Effect of TIM-3 Antibodies on Mouse Colon Cancer MC38 Subcutaneous Xenograft

Name of medicament to be tested:

TIM-3 antibody, h1799-005.

PD-1 antibody, murine PD-1 antibody J43 (J Immunol. 196(1):144-55.).

Experimental steps:

1×10⁶ mouse colon cancer MC38 cells were injected into the mouse's armpit. When the tumor was growing to an average volume of 50 to 200 mm³, the animals were randomly divided into groups according to the tumor volume, and administered. 40 mice were divided into 4 groups: negative control group (group 1), TIM-3 antibody, 30 mg/kg group (group 2), PD-1 antibody, 5 mg/kg group (group 3), and TIM-3 antibody in combination with PD-1 antibody group (group 4), 10 animals in each group; each group was administered with corresponding concentration of test substance via tail vein injection at a dosing volume of 10 ml/kg, and the dosing volume for the combined administration group was 20 mL/kg, twice per week, and administered for a period of 21 days.

Experimental results:

1. When compared with the tumor volume of 581±63 mm³ in negative control group, the tumor volumes for TIM-3 antibody 30 mg/kg group, PD-1 antibody 5 mg/kg group and combined administration group were 406±31 (P<0.05), 245±26 (P<0.01) and 166±19 (P<0.001) mm³, respectively, and significantly reduced;

2. When compared with the relative tumor volume (RTV) value of 5.38±0.56 in negative control group, the RTV values for TIM-3 antibody 30 mg/kg group, PD-1 antibody 5 mg/kg group and combined administration group were 3.76±0.32 (P<0.05), 2.20±0.21 (P<0.01) and 1.44±0.08 (P<0.001) respectively; T/C values were 69.91%, 40.92% and 26.66%, respectively;

3. When compared with the tumor weight of 0.3502±0.0298g in negative control group, the tumor weight for TIM-3 antibody 30 mg/kg group, PD-1 antibody 5 mg/kg group and combined administration group were 0.2550±0.0159 (P<0.01), 0.1820±0.0178 (P<0.001) and 0.1102±0.0106 g (P<0.001) respectively; IR were 27.19%, 48.05% and 65.36%, respectively;

4. The tumor volume, RTV value and tumor weight were analyzed. When compared with the TIM-3 antibody 30 mg/kg group, the combined administration group has significantly enhanced inhibition on tumor growth (P<0.001); when compared with the PD-1 antibody 5 mg/kg group, the combined administration group has significantly enhanced inhibition on tumor growth (P<0.05).

TABLE 8
The effect of antibodies on mouse colon cancer MC38 subcutaneous xenograft (x̅ ± SE)

| | dosage | Average weight (g) | | Tumor volume (mm³) | | | T/C |
|---|---|---|---|---|---|---|---|
| Group | mg/kg | D 1 | D 22 | D 1 | D 22 | RTV | (%) |
| 1 | 30 | 30.0 ± 0.5 | 31.0 ± 0.5 | 112 ± 9 | 581 ± 63 | 5.38 ± 0.56 | — |
| 2 | 30 | 29.9 ± 0.6 | 30.8 ± 0.5 | 112 ± 8 | 406 ± 31* | 3.76 ± 0.32* | 69.91 |
| 3 | 5 | 30.3 ± 0.4 | 30.8 ± 0.5 | 113 ± 8 | 245 ± 26** | 2.20 ± 0.21** | 40.92 |
| 4 | 30 + 5 | 30.9 ± 0.5 | 32.1 ± 0.6 | 113 ± 8 | 166 ± 19**^{###Δ} | 1.44 ± 0.08***^{###Δ} | 26.66 |

When compared with Group 1,
*P < 0.05,
**P < 0.01,
***P < 0.001.
When compared with Group 2,
^{###}P < 0.001.
When compared with Group 3,
^{Δ}P < 0.05.

TABLE 9
The effect of antibodies on tumor weight of mouse colon cancer
MC38 subcutaneous xenograft (x̅ ± SE)

| | dosage | Numbers of animals | | Tumor weight | IR |
|---|---|---|---|---|---|
| Group | mg/kg | D 1 | D 22 | (g) | (%) |
| 1 | 30 | 10 | 10 | 0.3502 ± 0.0298 | — |
| 2 | 30 | 10 | 10 | 0.2550 ± 0.0159** | 27.19 |
| 3 | 5 | 10 | 10 | 0.1820 ± 0.0.178*** | 48.05 |
| 4 | 30 + 5 | 10 | 10 | 0.1102 ± 0.0106***^{###Δ} | 65.36 |

When compared with Group 1,
* P < 0.05,
**P < 0.01,
***P < 0.001.
When compared with Group 2,
^{###}P < 0.001.
When compared with Group 3,
^{Δ}P < 0.05,
^{ΔΔ} P < 0.01,
^{ΔΔΔ} P < 0.001.

1. A method of treating a tumor in a patient in need thereof, the method comprising administering to the patient a therapeutically effective amount of a TIM-3 antibody or antigen-binding fragment thereof.

2. The method according to claim 1, wherein the TIM-3 antibody or antigen-binding fragment thereof comprises one or more CDR region sequences selected from the group consisting of:
antibody heavy chain variable region HCDR sequences as shown in amino acid sequence SEQ ID NOs: 14, 15 and 16, or amino acid sequences having at least 95% sequence identity thereto; and
antibody light chain variable region LCDR sequences as shown in amino acid sequence SEQ ID NOs: 17, 18 and 19, or amino acid sequences having at least 95% sequence identity thereto.

3. The method according to claim 2, wherein the TIM-3 antibody or antigen-binding fragment thereof is selected from the group consisting of murine antibody, chimeric antibody, humanized antibody or antigen-binding fragment thereof.

4. The method according to claim 3, wherein the humanized antibody comprises a light chain FR region and heavy chain FR region sequences derived from human germline light chain and heavy chain or mutant sequences thereof, respectively.

5. The method according to claim 3, wherein the humanized antibody comprises a heavy chain variable region as shown in SEQ ID NO: 31 or variant thereof and the humanized antibody comprises a light chain variable region as shown in SEQ ID NO: 32 or variant thereof.

6. The method according to claim 3, wherein the humanized antibody comprises a heavy chain variable region as shown in SEQ ID NO: 33 and a light chain variable region as shown in SEQ ID NO: 36.

7. The use method according to claim 1, wherein the TIM-3 antibody is a full-length antibody which further comprises human antibody constant region(s).

8. The method according to claim 1, wherein the antigen-binding fragment is selected from the group consisting of Fab, Fab′, F(ab′)2, single-chain antibody (scFv), dimerized V region (diabody), disulfide bond stabilized V region (dsFv), and antigen-binding fragment of peptide containing CDRs.

9. The method according to claim 1, wherein the tumor is selected from the group consisting of breast cancer, lung cancer, liver cancer, gastric cancer, colorectal cancer, kidney cancer, melanoma and non-small cell lung cancer.

10. The method according to claim 1, which is the use of the TIM-3 antibody or antigen-binding fragment thereof in combination with an anti-PD-1 antibody or antigen-binding fragment thereof for the preparation of a medicament for treating tumor.

11. The method according to claim 10, wherein the anti-PD-1 antibody or antigen-binding fragment thereof is humanized antibody or fragment thereof.

12. The method according to claim 10, wherein the antigen-binding fragment is selected from the group consisting of Fab, Fab′-SH, Fv, scFv and (Fab′)₂ fragment.

13. The method according to claim 11, wherein the anti-PD-1 antibody or antigen-binding fragment thereof comprises a heavy chain constant region of human IgG1, IgG2, IgG3 or IgG4 isotype.

14. The method according to claim 13, wherein the anti-PD-1 antibody or antigen-binding fragment thereof comprises a light chain constant region of kappa or lambda.

15. The method according to claim 11, wherein the anti-PD-1 antibody comprises a light chain variable region as shown in SEQ ID NO: 82 or variant thereof; and the anti-PD-1 antibody comprises a heavy chain variable region as shown in SEQ ID NO: 81 or variant thereof.

16. The method according to claim 11, wherein the anti-PD-1 antibody comprises a light chain as shown in SEQ ID NO: 80 or variant thereof; and the anti-PD-1 antibody comprises a heavy chain as shown in SEQ ID NO: 79 or variant thereof.

17. The method of claim 11, wherein the anti-PD-1 antibody comprises a light chain as shown in SEQ ID NO: 80 and a heavy chain as shown in SEQ ID NO: 79.

18. The method of claim 1, wherein the patient is a human, and wherein the TIM-3 antibody or antigen-binding fragment thereof is administered to the human at a dosage ranging from 0.1 mg/kg to 10.0 mg/kg.

19. The method according to claim 10, wherein the patient is a human, and wherein the anti-PD-1 antibody or antigen-binding fragment thereof is administered to the human at a dosage ranging from 0.1 mg/kg to 20.0 mg/kg.

20. A pharmaceutical composition, comprising an anti-TIM-3 antibody or antigen-binding fragment thereof, and an anti-PD-1 antibody or antigen-binding fragment thereof.


SEQUENCE LISTING (82 sequences)

SEQ ID NO: 1 - protein, 430 aa, Artificial Sequence
  Synthesized sequence Fusion of TIM-3 extracellular domain and hIgG1 Fc
  DOMAIN (1)..(430)
MEFGLSWLFLVAILKGVQCSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIREPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 215 aa, Artificial Sequence
  Synthesized sequence TIM-3 extracellular domain with Flag-tag, His-tag
  DOMAIN (1)..(215)
MEFGLSWLFLVAILKGVQCSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRGSSDYKDDDDKHHHHHH

SEQ ID NO: 3 - protein, 301 aa, Artificial Sequence
  Synthesized sequence TIM-3 full-length
  CHAIN (1)..(301)
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 4 - protein, 121 aa, Mus musculus
  DOMAIN (1)..(121): mAb-1701 antibody heavy chain variable region
EVQLQQSGPELVKPGASVKISCKASGYTFTDYYMNWVKQSHGKSLEWIADIIPNNGGSKYNQKFKDKATLTVDKSSSTAYMELRSLTSEDSAVYYCATWGYGSSYRWFDYWGQGTLVSVSA

SEQ ID NO: 5 - protein, 107 aa, Mus musculus
  DOMAIN (1)..(107): mAb-1701 antibody light chain variable region
DIQMTQSPASQSASLGESVTITCLASQPIGIWLAWYQQKPGKSPQLLIYAATSLADGVPSRFSGSGSGTKFSFKISSLQAEDFVSYYCQQLYSSPWTFGGGTKLEIK

SEQ ID NO: 6 - protein, 122 aa, Mus musculus
  DOMAIN (1)..(122): mAb-1799 antibody heavy chain variable region
EVKLVESEGGLVQPGSSMKLSCTASGFTFSDYYMAWVRQVPEKGLEWVANINYDGSSTYYLDSLKSRFIISRDNAKNILYLQMNSLKSDDTATYYCARDVGYYGGNYGFAYWGQGTLVTVSA

SEQ ID NO: 7 - protein, 107 aa, Mus musculus
  DOMAIN (1)..(107): mAb-1799 antibody light chain variable region
DIQMTQSPASLSASVGETVTITCRASDNIYSYLAWYQQKQGKSPQLLVYNAKTLAEGVPSRFSGSGSGTQFSLKINSLQPEDFGSYYCQQHYGSPLTFGAGTKLELK

SEQ ID NO: 8 - protein, 5 aa, Mus musculus
  DOMAIN (1)..(5): mAb-1701 antibody heavy chain HCDR1
DYYMN

SEQ ID NO: 9 - protein, 17 aa, Mus musculus
  DOMAIN (1)..(17): mAb-1701 antibody heavy chain HCDR2
DIIPNNGGSKYNQKFKD

SEQ ID NO: 10 - protein, 12 aa, Mus musculus
  DOMAIN (1)..(12): mAb-1701 antibody heavy chain HCDR3
WGYGSSYRWFDY

SEQ ID NO: 11 - protein, 11 aa, Mus musculus
  DOMAIN (1)..(11): mAb-1701 antibody light chain LCDR1
LASQPIGIWLA

SEQ ID NO: 12 - protein, 7 aa, Mus musculus
  DOMAIN (1)..(7): mAb-1701 antibody light chain LCDR2
AATSLAD

SEQ ID NO: 13 - protein, 9 aa, Mus musculus
  DOMAIN (1)..(9): mAb-1701 antibody light chain LCDR3
QQLYSSPWT

SEQ ID NO: 14 - protein, 5 aa, Mus musculus
  DOMAIN (1)..(5): mAb-1799 antibody heavy chain HCDR1
DYYMA

SEQ ID NO: 15 - protein, 17 aa, Mus musculus
  DOMAIN (1)..(17): mAb-1799 antibody heavy chain HCDR2
NINYDGSSTYYLDSLKS

SEQ ID NO: 16 - protein, 13 aa, Mus musculus
  DOMAIN (1)..(13): mAb-1799 antibody heavy chain HCDR3
DVGYYGGNYGFAY

SEQ ID NO: 17 - protein, 11 aa, Mus musculus
  DOMAIN (1)..(11): mAb-1799 antibody light chain LCDR1
RASDNIYSYLA

SEQ ID NO: 18 - protein, 7 aa, Mus musculus
  DOMAIN (1)..(7): mAb-1799 antibody light chain LCDR2
NAKTLAE

SEQ ID NO: 19 - protein, 9 aa, Mus musculus
  DOMAIN (1)..(9): mAb-1799 antibody light chain LCDR3
QQHYGSPLT

SEQ ID NO: 20 - protein, 121 aa, Artificial Sequence
  murine mAb-1701 humanized VH
  DOMAIN (1)..(121)
QVQLVQSGAEVKKPGASVKVSCKASGYTFTDYYMNWVRQAPGQGLEWMGDIIPNNGGSKYNQKFKDRVTMTTDTSTSTAYMELRSLRSDDTAVYYCARWGYGSSYRWFDYWGQGTLVTVSS

SEQ ID NO: 21 - protein, 107 aa, Artificial Sequence
  murine mAb-1701 humanized VL
  DOMAIN (1)..(107)
DIQMTQSPSSLSASVGDRVTITCLASQPIGIWLAWYQQKPGKAPKLLIYAATSLADGVPSRFSGSGSGTDFTFTISSLQPEDIATYYCQQLYSSPWTFGGGTKVEIK

SEQ ID NO: 22 - protein, 121 aa, Artificial Sequence; identical to SEQ ID NO: 20
  Synthesized sequence h1701_VH.1
  DOMAIN (1)..(121)

SEQ ID NO: 23 - protein, 121 aa, Artificial Sequence
  Synthesized sequence h1701h1701_VH.1A
  DOMAIN (1)..(121)
QVQLVQSGAEVKKPGASVKVSCKASGYTFTDYYMNWVRQAPGQGLEWIGDIIPNNGGSKYNQKFKDRVTMTTDTSTSTAYMELRSLRSDDTAVYYCARWGYGSSYRWFDYWGQGTLVTVSS

SEQ ID NO: 24 - protein, 121 aa, Artificial Sequence
  Synthesized sequence h1701_VH.1B
  DOMAIN (1)..(121)
QVQLVQSGAEVKKPGASVKVSCKASGYTFTDYYMNWVRQAPGQGLEWMGDIIPNNGGSKYNQKFKDRVTMTTDTSTSTAYMELRSLRSDDTAVYYCATWGYGSSYRWFDYWGQGTLVTVSS

SEQ ID NO: 25 - protein, 121 aa, Artificial Sequence
  Synthesized sequence h1701_VH.1C
  DOMAIN (1)..(121)
QVQLVQSGAEVKKPGASVKVSCKASGYTFTDYYMNWVRQAPGQGLEWIGDIIPNNGGSKYNQKFKDRVTMTTDTSTSTAYMELRSLRSDDTAVYYCATWGYGSSYRWFDYWGQGTLVTVSS

SEQ ID NO: 26 - protein, 121 aa, Artificial Sequence
  Synthesized sequence h1701_VH.1D
  DOMAIN (1)..(121)
QVQLVQSGAEVKKPGASVKVSCKASGYTFTDYYMNWVKQAPGQGLEWIGDIIPNNGGSKYNQKFKDRVTMTTDTSTSTAYMELRSLRSEDTAVYYCATWGYGSSYRWFDYWGQGTLVTVSS

SEQ ID NO: 27 - protein, 121 aa, Artificial Sequence
  Synthesized sequence h1701_VH.1E
  DOMAIN (1)..(121)
QVQLVQSGAEVKKPGASVKVSCKASGYTFTDYYMNWVRQAPGQGLEWIADIIPNNGGSKYNQKFKDRATLTTDTSTSTAYMELRSLRSDDTAVYYCATWGYGSSYRWFDYWGQGTLVTVSS

SEQ ID NO: 28 - protein, 121 aa, Artificial Sequence
  Synthesized sequence h1701_VH.1F
  DOMAIN (1)..(121)
QVQLVQSGAEVKKPGASVKVSCKASGYTFTDYYMNWVKQAPGQGLEWIADIIPNNGGSKYNQKFKDRATLTTDTSTSTAYMELRSLRSEDTAVYYCATWGYGSSYRWFDYWGQGTLVTVSS

SEQ ID NO: 29 - protein, 107 aa, Artificial Sequence; identical to SEQ ID NO: 21
  Synthesized sequence h1701_VL.1
  DOMAIN (1)..(107)

SEQ ID NO: 30 - protein, 107 aa, Artificial Sequence
  Synthesized sequence h1701_VL.1A
  DOMAIN (1)..(107)
DIQMTQSPSSLSASVGDRVTITCLASQPIGIWLAWYQQKPGKSPKLLIYAATSLADGVPSRFSGSGSGTDFTFTISSLQPEDIATYYCQQLYSSPWTFGGGTKVEIK

SEQ ID NO: 31 - protein, 122 aa, Artificial Sequence
  Synthesized sequence h1799VH-CDR graft
  DOMAIN (1)..(122)
EVQLVESGGGLVQPGGSLRLSCAASGFTFSDYYMAWVRQAPGKGLEWVANINYDGSSTYYLDSLKSRFTISRDNAKNSLYLQMNSLRAEDTAVYYCARDVGYYGGNYGFAYWGQGTLVTVSS

SEQ ID NO: 32 - protein, 107 aa, Artificial Sequence
  Synthesized sequence h1799VL-CDR graft
  DOMAIN (1)..(107)
DIQMTQSPSSLSASVGDRVTITCRASDNIYSYLAWYQQKPGKAPKLLIYNAKTLAEGVPSRFSGSGSGTDFTLTISSLQPEDFATYYCQQHYGSPLTFGQGTKLEIK

SEQ ID NO: 33 - protein, 122 aa, Artificial Sequence; identical to SEQ ID NO: 31
  Synthesized sequence h1799_VH.1
  DOMAIN (1)..(122)

SEQ ID NO: 34 - protein, 122 aa, Artificial Sequence
  Synthesized sequence h1799_VH.1A
  DOMAIN (1)..(122)
EVKLVESGGGLVQPGGSLRLSCAASGFTFSDYYMAWVRQAPGKGLEWVANINYDGSSTYYLDSLKSRFTISRDNAKNSLYLQMNSLRAEDTAVYYCARDVGYYGGNYGFAYWGQGTLVTVSS

SEQ ID NO: 35 - protein, 122 aa, Artificial Sequence
  Synthesized sequence h1799_VH.1B
  DOMAIN (1)..(122)
EVKLVESGGGLVQPGGSLRLSCAASGFTFSDYYMAWVRQAPGKGLEWVANINYDGSSTYYLDSLKSRFTISRDNAKNSLYLQMNSLKAEDTAVYYCARDVGYYGGNYGFAYWGQGTLVTVSS

SEQ ID NO: 36 - protein, 107 aa, Artificial Sequence; identical to SEQ ID NO: 32
  Synthesized sequence h1799_VL.1
  DOMAIN (1)..(107)

SEQ ID NO: 37 - protein, 107 aa, Artificial Sequence
  Synthesized sequence h1799_VL.1A
  DOMAIN (1)..(107)
DIQMTQSPSSLSASVGDRVTITCRASDNIYSYLAWYQQKPGKAPKLLVYNAKTLAEGVPSRFSGSGSGTDFTLTISSLQPEDFATYYCQQHYGSPLTFGQGTKLEIK

SEQ ID NO: 38 - protein, 107 aa, Artificial Sequence
  Synthesized sequence h1799_VL.1B
  DOMAIN (1)..(107)
DIQMTQSPSSLSASVGDRVTITCRASDNIYSYLAWYQQKPGKAPQLLVYNAKTLAEGVPSRFSGSGSGTDFTLTISSLQPEDFATYYCQQHYGSPLTFGQGTKLEIK

SEQ ID NO: 39 - protein, 107 aa, Artificial Sequence
  Synthesized sequence h1799_VL.1C
  DOMAIN (1)..(107)
DIQMTQSPSSLSASVGDRVTITCRASDNIYSYLAWYQQKPGKSPQLLVYNAKTLAEGVPSRFSGSGSGTDFTLTISSLQPEDFATYYCQQHYGSPLTFGQGTKLEIK

SEQ ID NO: 40 - protein, 107 aa, Artificial Sequence
  Synthesized sequence h1799_VL.1D
  DOMAIN (1)..(107)
DIQMTQSPSSLSASVGDRVTITCRASDNIYSYLAWYQQKPGKSPQLLVYNAKTLAEGVPSRFSGSGSGTDFTLTISSLQPEDFASYYCQQHYGSPLTFGQGTKLEIK

SEQ ID NO: 41 - protein, 327 aa, Artificial Sequence
  Synthesized sequence heavy chain constant region
  DOMAIN (1)..(327)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 42 - protein, 107 aa, Artificial Sequence [sequence omitted]

SEQ ID NO: 43 - protein, 17 aa, Artificial Sequence
  Synthesized sequence h1701 CDR2
  DOMAIN (5)..(5): Xaa may be Asn, Leu, Val, Met or Glu
  DOMAIN (6)..(6): Xaa may be Asn, Glu, Met, His, Lys, Leu, Ala or Val
  DOMAIN (7)..(7): Xaa may be Gly or Ala
DIIPXXXGSKYNQKFKD

SEQ ID NO: 44 - protein, 121 aa, Artificial Sequence
  Synthesized sequence h1701_VH.1-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVRQAPGQGLEWMGDIIPXXXGSKYNQKFKDRVTMTTDTSTSTAYMELRSLRSDDTAVYYCARWGYGSSYRWFDYWGQGTLVTVSS

SEQ ID NO: 45 - protein, 121 aa, Artificial Sequence
  Synthesized sequence h1701_VH.1A-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVRQAPGQGLEWIGDIIPXXXGSKYNQKFKDRVTMTTDTSTSTAYMELRSLRSDDTAVYYCARWGYGSSYRWFDYWGQGTLVTVSS

SEQ ID NO: 46 - protein, 121 aa, Artificial Sequence
  Synthesized sequence h1701_VH.1B-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVRQAPGQGLEWMGDIIPXXXGSKYNQKFKDRVTMTTDTSTSTAYMELRSLRSDDTAVYYCATWGYGSSYRWFDYWGQGTLVTVSS

SEQ ID NO: 47 - protein, 121 aa, Artificial Sequence
  Synthesized sequence h1701_VH.1C-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVRQAPGQGLEWIGDIIPXXXGSKYNQKFKDRVTMTTDTSTSTAYMELRSLRSDDTAVYYCATWGYGSSYRWFDYWGQGTLVTVSS

SEQ ID NO: 48 - protein, 121 aa, Artificial Sequence
  Synthesized sequence  h1701_VH.1D-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVKQAPGQGLEWIGDIIPXXXGSKYNQKFKDRVTMTTDTSTSTAYMELRSLRSEDTAVYYCATWGYGSSYRWFDYWGQGTLVTVSS

SEQ ID NO: 49 - protein, 121 aa, Artificial Sequence
  Synthesized sequence h1701_VH.1E-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVRQAPGQGLEWIADIIPXXXGSKYNQKFKDRATLTTDTSTSTAYMELRSLRSDDTAVYYCATWGYGSSYRWFDYWGQGTLVTVSS

SEQ ID NO: 50 - protein, 121 aa, Artificial Sequence
  Synthesized sequence h1701_VH.1F-CDR2
  DOMAIN (54)..(54): Xaa may be Asn, Leu, Val, Met or Glu
  DOMAIN (55)..(55): Xaa may be Asn, Glu, Met, His, Lys, Leu, Ala or Val
  DOMAIN (56)..(56): Xaa may be Gly or Ala
QVQLVQSGAEVKKPGASVKVSCKASGYTFTDYYMNWVKQAPGQGLEWIADIIPXXXGSKYNQKFKDRATLTTDTSTSTAYMELRSLRSEDTAVYYCATWGYGSSYRWFDYWGQGTLVTVSS

SEQ ID NO: 51 - protein, 121 aa, Artificial Sequence
  Synthesized sequence h1701-009NLG VH
  DOMAIN (1)..(121)
QVQLVQSGAEVKKPGASVKVSCKASGYTFTDYYMNWVRQAPGQGLEWMGDIIPNLGGSKYNQKFKDRVTMTTDTSTSTAYMELRSLRSDDTAVYYCATWGYGSSYRWFDYWGQGTLVTVSS

SEQ ID NO: 52 - protein, 121 aa, Artificial Sequence
  Synthesized sequence h1701-009NVG VH
  DOMAIN (1)..(121)
QVQLVQSGAEVKKPGASVKVSCKASGYTFTDYYMNWVRQAPGQGLEWMGDIIPNVGGSKYNQKFKDRVTMTTDTSTSTAYMELRSLRSDDTAVYYCATWGYGSSYRWFDYWGQGTLVTVSS

SEQ ID NO: 53 - protein, 121 aa, Artificial Sequence
  Synthesized sequence h1701-009NNA VH
  DOMAIN (1)..(121)
QVQLVQSGAEVKKPGASVKVSCKASGYTFTDYYMNWVRQAPGQGLEWMGDIIPNNAGSKYNQKFKDRVTMTTDTSTSTAYMELRSLRSDDTAVYYCATWGYGSSYRWFDYWGQGTLVTVSS

SEQ ID NO: 54 - protein, 121 aa, Artificial Sequence
  Synthesized sequence h1701-009NMA VH
  DOMAIN (1)..(121)
QVQLVQSGAEVKKPGASVKVSCKASGYTFTDYYMNWVRQAPGQGLEWMGDIIPNMAGSKYNQKFKDRVTMTTDTSTSTAYMELRSLRSDDTAVYYCATWGYGSSYRWFDYWGQGTLVTVSS

SEQ ID NO: 55 - protein, 121 aa, Artificial Sequence
  Synthesized sequence h1701-009NEA VH
  DOMAIN (1)..(121)
QVQLVQSGAEVKKPGASVKVSCKASGYTFTDYYMNWVRQAPGQGLEWMGDIIPNEAGSKYNQKFKDRVTMTTDTSTSTAYMELRSLRSDDTAVYYCATWGYGSSYRWFDYWGQGTLVTVSS

SEQ ID NO: 56 - protein, 121 aa, Artificial Sequence
  Synthesized sequence h1701-009NHA
  DOMAIN (1)..(121)
QVQLVQSGAEVKKPGASVKVSCKASGYTFTDYYMNWVRQAPGQGLEWMGDIIPNHAGSKYNQKFKDRVTMTTDTSTSTAYMELRSLRSDDTAVYYCATWGYGSSYRWFDYWGQGTLVTVSS

SEQ ID NO: 57 - protein, 121 aa, Artificial Sequence
  Synthesized sequence h1701-009NMG VH
  DOMAIN (1)..(121)
QVQLVQSGAEVKKPGASVKVSCKASGYTFTDYYMNWVRQAPGQGLEWMGDIIPNMGGSKYNQKFKDRVTMTTDTSTSTAYMELRSLRSDDTAVYYCATWGYGSSYRWFDYWGQGTLVTVSS

SEQ ID NO: 58 - protein, 121 aa, Artificial Sequence
  Synthesized sequence h1701-009NEG VH
  DOMAIN (1)..(121)
QVQLVQSGAEVKKPGASVKVSCKASGYTFTDYYMNWVRQAPGQGLEWMGDIIPNEGGSKYNQKFKDRVTMTTDTSTSTAYMELRSLRSDDTAVYYCATWGYGSSYRWFDYWGQGTLVTVSS

SEQ ID NO: 59 - protein, 121 aa, Artificial Sequence
  Synthesized sequence h1701-009NKG VH
  DOMAIN (1)..(121)
QVQLVQSGAEVKKPGASVKVSCKASGYTFTDYYMNWVRQAPGQGLEWMGDIIPNKGGSKYNQKFKDRVTMTTDTSTSTAYMELRSLRSDDTAVYYCATWGYGSSYRWFDYWGQGTLVTVSS

SEQ ID NO: 60 - protein, 121 aa, Artificial Sequence
  Synthesized sequence h1701-009NAG VH
  DOMAIN (1)..(121)
QVQLVQSGAEVKKPGASVKVSCKASGYTFTDYYMNWVRQAPGQGLEWMGDIIPNAGGSKYNQKFKDRVTMTTDTSTSTAYMELRSLRSDDTAVYYCATWGYGSSYRWFDYWGQGTLVTVSS

SEQ ID NO: 61 - protein, 121 aa, Artificial Sequence
  Synthesized sequence h1701-009NHG VH
  DOMAIN (1)..(121)
QVQLVQSGAEVKKPGASVKVSCKASGYTFTDYYMNWVRQAPGQGLEWMGDIIPNHGGSKYNQKFKDRVTMTTDTSTSTAYMELRSLRSDDTAVYYCATWGYGSSYRWFDYWGQGTLVTVSS

SEQ ID NO: 62 - protein, 17 aa, Artificial Sequence
  Synthesized sequence h1701-009NLG HCDR2
  DOMAIN (1)..(17)
DIIPNLGGSKYNQKFKD

SEQ ID NO: 63 - protein, 17 aa, Artificial Sequence
  Synthesized sequence h1701-009NVG HCDR2
  DOMAIN (1)..(17)
DIIPNVGGSKYNQKFKD

SEQ ID NO: 64 - protein, 17 aa, Artificial Sequence
  Synthesized sequence h1701-009NNA HCDR2
  DOMAIN (1)..(17)
DIIPNNAGSKYNQKFKD

SEQ ID NO: 65 - protein, 17 aa, Artificial Sequence
  Synthesized sequence h1701-009NMA HCDR2
  DOMAIN (1)..(17)
DIIPNMAGSKYNQKFKD

SEQ ID NO: 66 - protein, 17 aa, Artificial Sequence
  Synthesized sequence h1701-009NEA HCDR2
  DOMAIN (1)..(17)
DIIPNEAGSKYNQKFKD

SEQ ID NO: 67 - protein, 17 aa, Artificial Sequence
  Synthesized sequence h1701-009NHA HCDR2
  DOMAIN (1)..(17)
DIIPNHAGSKYNQKFKD

SEQ ID NO: 68 - protein, 17 aa, Artificial Sequence
  Synthesized sequence h1701-009NMG HCDR2
  DOMAIN (1)..(17)
DIIPNMGGSKYNQKFKD

SEQ ID NO: 69 - protein, 17 aa, Artificial Sequence
  Synthesized sequence h1701-009NEG HCDR2
  DOMAIN (1)..(17)
DIIPNEGGSKYNQKFKD

SEQ ID NO: 70 - protein, 17 aa, Artificial Sequence
  Synthesized sequence h1701-009NKG HCDR2
  DOMAIN (1)..(17)
DIIPNKGGSKYNQKFKD

SEQ ID NO: 71 - protein, 17 aa, Artificial Sequence
  Synthesized sequence h1701-009NAG HCDR2
  DOMAIN (1)..(17)
DIIPNAGGSKYNQKFKD

SEQ ID NO: 72 - protein, 17 aa, Artificial Sequence
  Synthesized sequence h1701-009NHG HCDR2
  DOMAIN (1)..(17)
DIIPNHGGSKYNQKFKD

SEQ ID NO: 73 - protein, 5 aa, Mus musculus
  DOMAIN (1)..(5): anti PD-1 antibody HCDR1
SYMMS

SEQ ID NO: 74 - protein, 17 aa, Mus musculus
  DOMAIN (1)..(17): anti PD-1 antibody HCDR2
TISGGGANTYYPDSVKG

SEQ ID NO: 75 - protein, 7 aa, Mus musculus
  DOMAIN (1)..(7): anti PD-1 antibody HCDR3
QLYYFDY

SEQ ID NO: 76 - protein, 11 aa, Mus musculus
  DOMAIN (1)..(11): anti PD-1 antibody LCDR1
LASQTIGTWLT

SEQ ID NO: 77 - protein, 7 aa, Mus musculus
  DOMAIN (1)..(7): anti PD-1 antibody LCDR2
TATSLAD

SEQ ID NO: 78 - protein, 9 aa, Mus musculus
  DOMAIN (1)..(9): anti PD-1 antibody LCDR3
QQVYSIPWT

SEQ ID NO: 79 - protein, 443 aa, Artificial Sequence
  Humanized anti PD-1 antibody  heavy chain
  CHAIN (1)..(443)
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 80 - protein, 214 aa, Artificial Sequence
  Humanized anti PD-1 antibody light chain
  CHAIN (1)..(214)
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 81 - protein, 116 aa, Artificial Sequence
  Humanized anti PD-1 antibody heavy chain variable region
  DOMAIN (1)..(116)
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 82 - protein, 107 aa, Artificial Sequence
  Humanized anti PD-1 antibody light chain variable region
  DOMAIN (1)..(107)
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK